(19) United States
(12) Patent Application Publication
(10) Pub. No.: US 2024/0324970 A1
(43) Pub. Date: Oct. 3, 2024

(54) SENSING RESPIRATION PARAMETERS AS INDICATOR OF SUDDEN CARDIAC ARREST EVENT

(71) Applicant: Medtronic, Inc., Minneapolis, MN (US)

(72) Inventors: Shantanu Sarkar, Roseville, MN (US); Paul G. Krause, Mahtomedi, MN (US); Kevin T. Ousdigian, Shoreview, MN (US); Paul J. DeGroot, Minneapolis, MN (US)

(21) Appl. No.: 18/552,324

(22) PCT Filed: Feb. 17, 2022
(86) PCT No.: PCT/US2022/016680

§ 371 Date: Sep. 25, 2023

Related U.S. Application Data
(60) Provisional application No. 63/182,512, filed on Apr. 30, 2021.

(51) Int. Cl. A61B 5/00; A61B 5/0205; A61B 5/08; A61B 5/11; A61B 5/113; A61B 5/29; A61B 5/296; A61B 5/346; A61B 5/397

(52) U.S. Cl. CPC A61B 5/7282; A61B 5/0002; A61B 5/0205; A61B 5/1117; A61B 5/29; A61B 5/296; A61B 5/346; A61B 5/397; A61B 5/686; A61B 5/7221; A61B 5/7239; A61B 5/7285; A61B 5/746; A61B 5/747; A61B 5/0809; A61B 5/0816; A61B 5/113; A61B 2560/045; A61B 2560/0468; A61B 2562/0204; A61B 2562/0219

(57) ABSTRACT

Devices, systems, and techniques for detecting a sudden cardiac event based on respiratory parameter information. A method includes receiving periodic respiratory parameter information, where the respiratory parameter information includes respiratory effort of a patient; and determining, by the processing circuitry and based on the respiratory parameter information, whether a sudden cardiac arrest of the patient is detected.

### TECHNICAL FIELD

The disclosure relates generally to medical device systems and, more particularly, medical device systems configured to monitor patient parameters.

### BACKGROUND

Some types of medical devices may be used to monitor one or more physiological parameters of a patient. Such medical devices may include, or may be part of a system that includes, sensors that detect signals associated with such physiological parameters. Values determined based on such signals may be used to assist in detecting changes in patient conditions, in evaluating the efficacy of a therapy, or in generally evaluating patient health.

### SUMMARY

In general, the disclosure is directed to devices, systems, and techniques for using a medical device to perform a measurement indicative of a respiratory parameter of a patient to predict or confirm a sudden cardiac arrest (SCA) event. SCA events may be associated with lack of breathing, rapid breathing, deep breathing, labored breathing, gasping, agonal breathing, or other abnormal breathing. Measurement of respiratory parameters according to the techniques described herein may provide a technical improvement in the ability of a device or system to detect SCA events, e.g., sensitivity and specificity of algorithms employed to detect SCA events.

The measurement may include one or more of an impedance signal, accelerometer signal, or electromyography (EMG) signal. The impedance of an electrical path between electrodes of the medical device, in some cases, may represent a resistance associated with contact between the electrodes and target tissue of the patient, and/or the impedance of the tissue in the path between the electrodes. As such, impedance may change over a period of time according to movements of the patient, such as movement of the patient's thorax, and/or changes in the impedance of patient tissue. For example, as the patient's chest cavity moves during a respiration cycle, contact between the electrodes and the target tissue may change, thus causing impedance to change. Furthermore, the relative fluid content of the tissue within the path may change during the respiration cycle. The accelerometer signal may indicate whether an accelerometer on the chest if the patient's thorax has moved. The EMG signal may represent electrical activity associated with movement of muscle contraction or activation of muscle. For example, as the patient's chest cavity moves during a respiration cycle, an accelerometer signal or EMG signal changes.

In some cases, the signal may change according to a periodic function corresponding to respiratory cycles (e.g., breathing in and breathing out) performed by the patient. In this way, processing circuitry may analyze a signal obtained by the medical device to identify parameters associated with the patient's respiratory cycles, such as respiratory rate, respiratory rate variability, and respiratory effort, as examples. Such parameters may in turn be analyzed, e.g., by the processing circuitry and/or artificial intelligence, to confirm or predict a SCA event.

The medical device may, in some examples, perform a set of measurements. In some cases, the medical device may perform the set of measurements at a measurement rate. In this way, the set of measurements may present a detailed picture of the patient's respiratory patterns over an extended period of time, enabling processing circuitry to identify trends in the respiration parameter data or otherwise analyze the data to identify or monitor the patient conditions. Although, in some cases, the medical device may be configured consistently to perform the set of measurements at the measurement rate, in other cases, the medical device may measure one or more patient parameters upon detection of an event. Additionally, in some examples, the medical device may determine whether to perform measurements based on a heart rate, a patient posture (e.g., sitting, standing, or laying down), an electrocardiogram (ECG), a presence or an absence of one or more arrhythmias, patient triggers, or presence of a suspected sudden cardiac arrest.

To determine respiratory parameter information of the patient during a particular measurement, processing circuitry may be configured to process the signal corresponding to the measurement to identify a set of respiratory intervals. Each respiratory interval of the set of respiratory intervals may represent a full respiratory cycle (e.g., a combination of an exhaling phase and an inhaling phase).

In one example, this disclosure describes a method includes receiving, by a processing circuitry, periodic respiratory parameter information, where the respiratory parameter information includes respiratory effort of a patient; and determining, by the processing circuitry and based on the respiratory parameter information, whether a sudden cardiac arrest of the patient is detected.

In another example, this disclosure describes a device includes processing circuitry and a memory comprising program instructions that, when executed by the processing circuitry, cause the processing circuitry to receive periodic respiratory parameter information, where the respiratory parameter information includes respiratory effort of a patient, and determine, based on the respiratory parameter information, whether a sudden cardiac arrest of the patient is detected.

In another example, this disclosure describes a computer-readable storage medium includes receive, by a processing circuitry, periodic respiratory parameter information, where the respiratory parameter information includes respiratory effort of a patient; and determine, by the processing circuitry and based on the respiratory parameter information, whether a sudden cardiac arrest of the patient is detected.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a block diagram illustrating an example system configured detect acute health events of a patient, and to respond to such detections, in accordance with one or more techniques of this disclosure.

FIG. 2 is a conceptual drawing illustrating an example configuration of the implantable medical device (IMD) of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIG. 4 is block diagram illustrating an example configuration of a computing device that operates in accordance with one or more techniques of the present disclosure.

FIG. 5 is a block diagram illustrating an example configuration of a health monitoring system that operates in accordance with one or more techniques of the present disclosure.

FIG. 6 is a flow diagram illustrating an example operation for performing parameter measurements, in accordance with one or more techniques of this disclosure.

FIG. 7 is a flow diagram illustrating an example operation for processing a set of values, in accordance with one or more techniques of this disclosure.

FIG. 8 is a flow diagram illustrating an example operation for determining a set of positive zero crossings in a measurement, in accordance with one or more techniques of this disclosure.

FIG. 9 is a flow diagram illustrating an example operation for determining a set of negative zero crossings in a measurement, in accordance with one or more techniques of this disclosure.

FIG. 10 is a flow diagram illustrating an example operation for determining a set of respiration intervals, in accordance with one or more techniques of this disclosure.

FIG. 11 is a flow diagram illustrating an example operation for determining a peak-to-peak value, in accordance with one or more techniques of this disclosure.

FIG. 12 is a flow diagram illustrating an example operation for determining a quality of a measurement, in accordance with one or more techniques of this disclosure.

FIG. 13 is a flow diagram illustrating an example operation for determining respiratory parameter information, in accordance with one or more techniques of this disclosure.

FIG. 14 is a flow diagram illustrating an example operation for determining respiratory parameter information, in accordance with one or more techniques of this disclosure.

Like reference characters denote like elements throughout the description and figures.

### DETAILED DESCRIPTION

A variety of types of implantable and external devices are configured to detect arrhythmia episodes and other acute health events based on sensed ECGs and, in some cases, other physiological signals. External devices that may be used to non-invasively sense and monitor ECGs and other physiological signals include wearable devices with electrodes configured to contact the skin of the patient, such as patches, watches, or necklaces. Such external devices may facilitate relatively longer-term monitoring of patient health during normal daily activities.

Implantable medical devices (EVIDs) also sense and monitor ECGs and other physiological signals, and detect acute health events such as episodes of arrhythmia, cardiac arrest, myocardial infarction, stroke, and seizure. Example EVIDs include pacemakers and implantable cardioverter-defibrillators, which may be coupled to intravascular or extravascular leads, as well as pacemakers with housings configured for implantation within the heart, which may be leadless. Some EVIDs do not provide therapy, such as implantable patient monitors. One example of such an IMD is the Reveal LINQ II™ Insertable Cardiac Monitor (ICM), available from Medtronic plc, which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

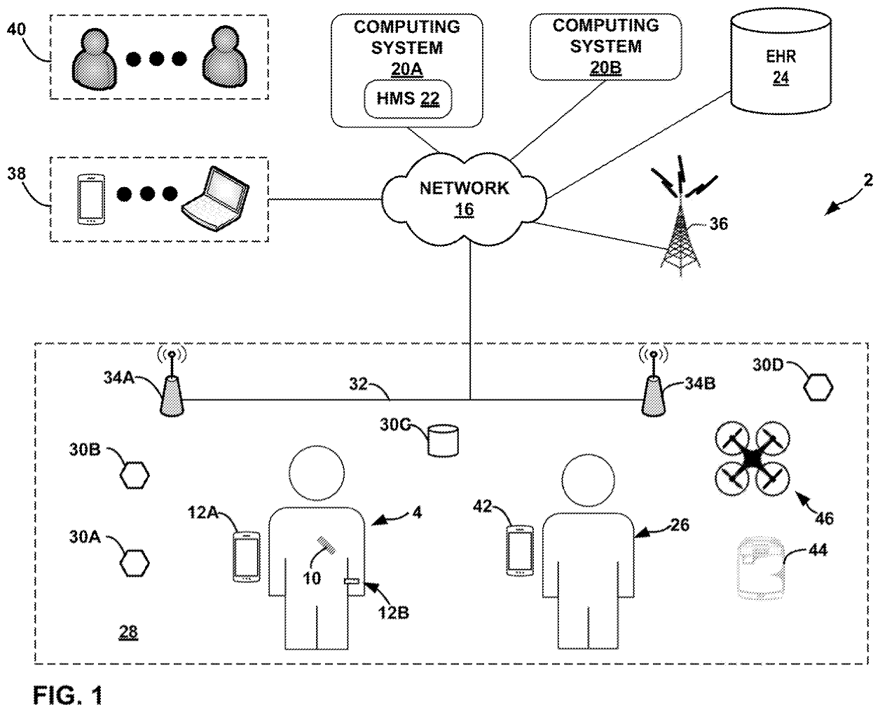

FIG. 1 is a block diagram illustrating an example system 2 configured detect acute health events of a patient 4, and to respond to such detection, in accordance with one or more techniques of this disclosure. As used herein, the terms “detect,” “detection,” and the like may refer to detection of an acute health event presently (at the time the data is collected) being experienced by patient 4, as well as detection based on the data that the condition of patient 4 is such that they have a suprathreshold likelihood of experiencing the event within a particular timeframe, e.g., prediction of the acute health event. The example techniques may be used with one or more patient sensing devices, e.g., IMD 10, which may be in wireless communication with one or more patient computing devices, e.g., patient computing devices 12A and 12B (collectively, “patient computing devices 12”). Although not illustrated in FIG. 1, IMD 10 include electrodes and other sensors to sense physiological signals of patient 4, and may collect and store sensed physiological data based on the signals and detect episodes based on the data.

IMD 10 may be implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. In some examples, IMD 10 takes the form of the LINQ II™ ICM. Although described primarily in the context of examples in which IMD 10 takes the form of an ICM, the techniques of this disclosure may be implemented in systems including any one or more implantable or external medical devices, including monitors, pacemakers, defibrillators, wearable external defibrillators, neurostimulators, or drug pumps. Furthermore, although described primarily in the context of examples including a single implanted patient sensing device, in some examples a system includes one or more patient sensing devices, which may be implanted within patient 4 or external to (e.g., worn by) patient 4.

Patient computing devices 12 are configured for wireless communication with IMD 10. Computing devices 12 retrieve event data and other sensed physiological data from IMD 10 that was collected and stored by the IMD. In some examples, computing devices 12 take the form of personal computing devices of patient 4. For example, computing device 12A may take the form of a smartphone of patient 4, and computing device 12B may take the form of a smartwatch or other smart apparel of patient 4. In some examples, computing devices 12 may be any computing device configured for wireless communication with IMD 10, such as a desktop, laptop, or tablet computer. Computing devices 12 may communicate with IMD 10 and each other according to the Bluetooth® or Bluetooth® Low Energy (BLE) protocols, as examples. In some examples, only one of computing devices 12, e.g., computing device 12A, is configured for communication with IMD 10, e.g., due to execution of software (e.g., part of a health monitoring application as described herein) enabling communication and interaction with an ID.

In some examples, computing device(s) 12, e.g., wearable computing device 12B in the example illustrated by FIG. 1, may include electrodes and other sensors to sense physiological signals of patient 4, and may collect and store physiological data and detect episodes based on such signals. Computing device 12B may be incorporated into the apparel of patient 14, such as within clothing, shoes, eyeglasses, a watch or wristband, a hat, etc. In some examples, computing device 12B is a smartwatch or other accessory or peripheral for a smartphone computing device 12A. In addition, computing device 12B may include peripheral devices which may estimate respiratory rate and/or effort. In some examples, the peripheral devices include radar based systems, LiDAR sensor systems, MAX Low Light systems, depth sensing camera systems, acoustic sensor systems, or pulse oximetry systems.

One or more of computing devices 12 may be configured to communicate with a variety of other devices or systems via a network 16. For example, one or more of computing devices 12 may be configured to communicate with one or more computing systems, e.g., computing systems 20A and 20B (collectively, “computing systems 20”) via network 16. Computing systems 20A and 20B may be respectively managed by manufacturers of IMD 10 and computing devices 12 to, for example, provide cloud storage and analysis of collected data, maintenance and software services, or other networked functionality for their respective devices and users thereof. Computing system 20A may comprise, or may be implemented by, the Medtronic Carelink™ Network, in some examples. In the example illustrated by FIG. 1, computing system 20A implements a health monitoring system (HMS) 22, although in other examples, either of both of computing systems 20 may implement HMS 22. As will be described in greater detail below, HMS 22 facilities detection of acute health events of patient 4 by system 2, and the responses of system 2 to such acute health events.

Computing device(s) 12 may transmit data, including data retrieved from IMD 10, to computing system(s) 20 via network 16. The data may include sensed data, e.g., values of physiological parameters measured by IMD 10 and, in some cases one or more of computing devices 12, data regarding episodes of arrhythmia or other acute health events detected by IMD 10 and computing device(s) 12, and other physiological signals or data recorded by IMD 10 and/or computing device(s) 12. HMS 22 may also retrieve data regarding patient 4 from one or more sources of electronic health records (EHR) 24 via network. EHR 24 may include data regarding historical (e.g., baseline) physiological parameter values, previous health events and treatments, disease states, comorbidities, demographics, height, weight, and body mass index (BMI), as examples, of patients including patient 4. HMS 22 may use data from EHR 24 to configure algorithms implemented by IMD 10 and/or computing devices 12 to detect acute health events for patient 4. In some examples, HMS 22 provides data from EHR 24 to computing device(s) 12 and/or IMD 10 for storage therein and use as part of their algorithms for detecting acute health events.

Network 16 may include one or more computing devices, such as one or more non-edge switches, routers, hubs, gateways, security devices such as firewalls, intrusion detection, and/or intrusion prevention devices, servers, cellular base stations and nodes, wireless access points, bridges, cable modems, application accelerators, or other network devices. Network 16 may include one or more networks administered by service providers, and may thus form part of a large-scale public network infrastructure, e.g., the Internet. Network 16 may provide computing devices and systems, such as those illustrated in FIG. 1, access to the Internet, and may provide a communication framework that allows the computing devices and systems to communicate with one another. In some examples, network 16 may include a private network that provides a communication framework that allows the computing devices and systems illustrated in FIG. 1 to communicate with each other, but isolates some of the data flows from devices external to the private network for security purposes. In some examples, the communications between the computing devices and systems illustrated in FIG. 1 are encrypted.

As will be described herein, ID 10 may be configured to detect acute health events of patient 4 based on data sensed by IMD 10 and, in some cases, other data, such as data sensed by computing devices 12A and/or 12B, and data from EHR 24. In response to detection of an acute health event, IMD 10 may wirelessly transmit a message to one or both of computing devices 12A and 12B. The message may indicate that IMD 10 detected an acute health event of the patient. The message may indicate a time that IMD 10 detected the acute health event. The message may include physiological data collected by ID 10, e.g., data which lead to detection of the acute health event, data prior to detection of the acute health event, and/or real-time or more recent data collected after detection of the acute health event. The physiological data may include values of one or more physiological parameters and/or digitized physiological signals. Examples of acute health events are a cardiac arrest, a ventricular fibrillation, a ventricular tachycardia, myocardial infarction, a pause in heart rhythm (asystole), or Pulseless Electrical Activity (PEA), acute respiratory distress syndrome (ARDS), a stroke, a seizure, or a fall.

In response to the message from IMD 10, computing device(s) 12 may output an alarm that may be visual and/or audible, and configured to immediately attract the attention of patient 4 or any person in environment 28 with patient 4, e.g., a bystander 26. Environment 28 may be a home, office, or place of business, or public venue, as examples. Computing device(s) 12 may also transmit a message to HMS 22 via network 16. The message may include the data received from IMD 10 and, in some cases, additional data collected by computing device(s) 12 or other devices in response to the detection of the acute health event by IMD 10. For example, the message may include a location of patient 4 determined by computing device(s) 12.

Other devices in the environment 28 of patient 4 may also be configured to output alarms, or alerts or take other actions to attract the attention of patient 4 and, possibly, a bystander 26, or to otherwise facilitate the delivery of care to patient 4. For example, environment 28 may include one or more Internet of Things (IoT) devices, such as IoT devices 30A-30D (collectively “IoT devices 30”) illustrated in the example of FIG. 1. IoT devices 30 may include, as examples, so called “smart” speakers, cameras, lights, locks, thermostats, appliances, actuators, controllers, or any other smart home (or building) devices. In the example of FIG. 1, IoT device 30C is a smart speaker and/or controller, which may include a display. IoT devices 30 may provide audible and/or visual alarms when configured with output devices to do so. As other examples, IoT devices 30 may cause smart lights throughout environment 28 to flash or blink and unlock doors. In some examples, IoT devices 30 that include cameras or other sensors may activate those sensors to collect data regarding patient 4, e.g., for evaluation of the condition of patient 4.

Computing device(s) 12 may be configured to wirelessly communicate with IoT devices 30 to cause IoT devices 30 to take the actions described herein. In some examples, HMS 22 communicates with IoT devices 30 via network 16 to cause IoT devices 30 to take the actions described herein, e.g., in response to receiving the alert message from computing device(s) 12 as described above. In some examples, IMD 10 is configured to communicate wirelessly with one or more of IoT devices 30, e.g., in response to detection of an acute health event when communication with computing devices 12 is unavailable. In such examples, IoT device(s) 30 may be configured to provide some or all of the functionality ascribed to computing devices 12 herein.

Environment 28 includes computing facilities, e.g., a local network 32, by which computing devices 12, IoT devices 30, and other devices within environment 28 may communicate via network 16, e.g., with HMS 22. For example, environment 28 may be configured with wireless technology, such as IEEE 802.11 wireless networks, IEEE 802.15 ZigBee networks, an ultra-wideband protocol, near-field communication, or the like. Environment 28 may include one or more wireless access points, e.g., wireless access points 34A and 34B (collectively, “wireless access points 34”) that provide support for wireless communications throughout environment 28. Additionally, or alternatively, e.g., when local network is unavailable, computing devices 12, IoT devices 30, and other devices within environment 28 may be configured to communicate with network 16, e.g., with HMS 22, via a cellular base station 36 and a cellular network.

Computing device(s) 12, and in some examples IoT devices 30, may include input devices and interfaces to allow a user to override the alarm in the event the detection of the acute health event by IMD 10 was false. In some examples, one or more of computing device(s) 12 and IoT device(s) 30 may implement an event assistant. The event assistant may provide a conversational interface for patient 4 and/or bystander 26 to exchange information with the computing device or IoT device. The event assistant may query the user regarding the condition of patient 4 in response to receiving the alert message from IMD 10. Responses from the user may be used to confirm or override detection of the acute health event by IMD 10, or to provide additional information about the acute health event or the condition of patient 4 more generally that may improve the efficacy of the treatment of patient 4. For example, information received by the event assistant may be used to provide an indication of severity or type (differential diagnosis) for the acute health event. The event assistant may use natural language processing and context data to interpret utterances by the user. In some examples, in addition to receiving responses to queries posed by the assistant, the event assistant may be configured to respond to queries posed by the user. For example, patient 4 may indicate that they feel dizzy and ask the event assistant, “how am I doing?”.

In some examples, computing device(s) 12 and/or HMS 22 may implement one or more algorithms to evaluate the sensed physiological data received from ID 10, and in some cases additional physiological or other data sensed or otherwise collected by the computing device(s) or IoT devices 30, to confirm or override the detection of the acute health event by IMD 10. In some examples, computing device(s) 12 and/or computing system(s) 20 may have greater processing capacity than IMD 10, enabling more complex analysis of the data. In some examples, the computing device(s) 12 and/or HMS 22 may apply the data to a machine learning model or other artificial intelligence developed algorithm, e.g., to determine whether the data is sufficiently indicative of the acute health event.

In examples in which computing device(s) 12 are configured to perform an acute health event confirmation analysis, computing device(s) 12 may transmit alert messages to HMS 22 and/or IoT devices 30 in response to confirming the acute health event. In some examples, computing device(s) 12 may be configured to transmit the alert messages prior to completing the confirmation analysis, and transmit cancellation messages in response to the analysis overriding the detection of the acute health event by IMD 10. HMS 22 may be configured to perform a number of operations in response to receiving an alert message from computing device(s) 12 and/or IoT device(s) 30. HMS 22 may be configured to cancel such operations in response to receiving a cancellation message from computing device(s) 12 and/or IoT device(s) 30.

For example, HMS 22 may be configured to transmit alert messages to one or computing devices 38 associated with one or more care providers 40 via network 16. Care providers may include emergency medical systems (EMS) and hospitals, and may include particular departments within a hospital, such as an emergency department, catheterization lab, or a stroke response department. Computing devices 38 may include smartphones, desktop, laptop, or tablet computers, or workstations associated with such systems or entities, or employees of such systems or entities. The alert messages may include any of the data collected by IMD 10, computing device(s) 12, and IoT device(s) 30, including sensed physiological data, time of the acute health event, location of patient 4, and results of the analysis by IMD 10, computing device(s) 12, IoT device(s) 30, and/or HMS 22. The information transmitted from HMS 22 to care providers 40 may improve the timeliness and effectiveness of treatment of the acute health event of patient 4 by care providers 40. In some examples, instead of or in addition to HMS 22 providing an alert message to one or more computing devices 38 associated with an EMS care provider 40, computing device(s) 12 and/or IoT devices 30 may be configured to automatically contact EMS, e.g., in the United States/North America, use the telephone system to contact a 911 call center, in response to receiving an alert message from IMD 10. Again, such operations may be cancelled by patient 4, bystander 26, or another user via a user interface of computing device(s) 12 or IoT device(s) 30, or automatically cancelled by computing device(s) 12 based on a confirmatory analysis performed by the computing device(s) overriding the detection of the acute health event by IMD 10.

Similarly, HMS 22 may be configured to transmit an alert message to computing device 42 of bystander 26, which may improve the timeliness and effectiveness of treatment of the acute health event of patient 4 by bystander 26. Computing device 42 may be similar to computing devices 12 and computing devices 38, e.g., a smartphone. In some examples, HMS 22 may determine that bystander 26 is proximate to patient 4 based on a location of patient 4, e.g., received from computing device(s) 12, and a location of computing device 42, e.g., reported to HMS 22 by an application implemented on computing device 42. In some examples, HMS 22 may transmit the alert message to any computing devices 42 in an alert area determined based on the location of patient 4, e.g., by transmitting the alert message to all computing devices in communication with base station 36.

In some examples, the alert message to bystander 26 may be configured to assist a layperson in treating patient. For example, the alert message to bystander 26 may include a location (and in some cases a description) of patient 4, the general nature of the acute health event, directions for providing care to patient 4, such as directions for providing cardio-pulmonary resuscitation (CPR), a location of nearby medical equipment for treatment of patient 4, such as an automated external defibrillator (AED) 44 or a life vest, and instructions for use of the equipment. In some examples, computing device(s) 12, IoT device(s) 30, and/or computing device 42 may implement an event assistant configured to use natural language processing and context data to provide a conversational interface for bystander 42. The assistant may provide bystander 26 with directions for providing care to patient 4, and respond to queries from bystander 26 about how to provide care to patient 4.

In some examples, HMS 22 may mediate bi-directional audio (and in some cases video) communication between care providers 40 and patient 4 or bystander 26. Such communication may allow care providers 40 to evaluate the condition of patient 4, e.g., through communication with patient 4 or bystander 26, or through use of a camera or other sensors of the computing device or IoT device, in advance of the time they will begin caring for the patient, which may improve the efficacy of care delivered to the patient. Such communication may also allow the care providers to instruct bystander 42 regarding first responder treatment of patient 4.

In some examples, HMS 22 may control dispatch of a drone 46 to environment 28, or a location near environment 28 or patient 4. Drone 46 may be a robot and/or unmanned aerial vehicle (UAV). Drone 46 may be equipped with a number of sensors and/or actuators to perform a number of operations. For example, drone 46 may include a camera or other sensors to navigate to its intended location, identify patient 4 and, in some cases, bystander 26, and to evaluate a condition of patient. In some examples, drone 46 may include user interface devices to communicate with patient 4 and/or bystander 26. In some examples, drone 46 may provide directions to bystander 26, to the location of patient 4 and regarding how to provide first responder care, such as CPR, to patient 4. In some examples, drone 46 may carry medical equipment, e.g., AED 44, and/or medication to the location of patient 4. In some examples, drone 46 may perform a ECG or pulse measurement. In some examples, drone 46 may act as an AED, for example, by touching two parts of a patient body, for example with extendable members which contain electrodes.

As will be described in greater detail below, IMD 10 or another device of system 2 may be configured to sense a signal that varies with respiration of patient 4, e.g., an impedance, accelerometer, or EMG signal, ECG signal, sound signals, or optical signals. Processing circuitry of system 2, e.g., of IMD 10 or computing device(s) 12 may receive periodic respiratory parameter information determined based on the signal, and determine whether an SCA event of patient 4 is detected based on the signal. In some examples, the processing circuitry uses the respiratory parameter information for an initial detection of the SCA event, determine urgency required for a response for the SCA event, and/or for a confirmation (or rejection) of a detection of the SCA event based on one or more other patient parameters.

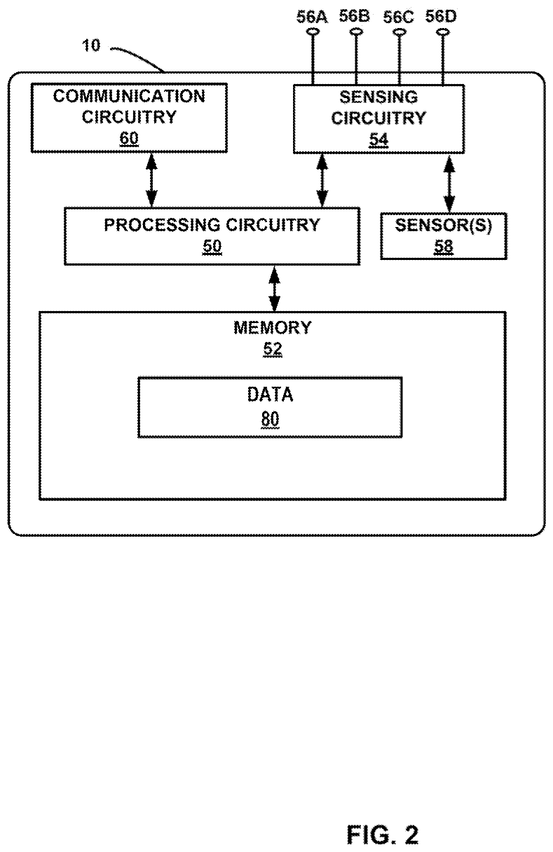

FIG. 2 is a block diagram illustrating an example configuration of IMD 10 of FIG. 1. As shown in FIG. 2, ID 10 includes processing circuitry 50, memory 52, sensing circuitry 54 coupled to electrodes 56A and 56B (hereinafter, “electrodes 56”) and one or more sensor(s) 58, and communication circuitry 60.

Processing circuitry 50 may include fixed function circuitry and/or programmable processing circuitry. Processing circuitry 50 may include any one or more of a microprocessor, a controller, a graphics processing unit (GPU), a tensor processing unit (TPU), a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more GPUs, one or more TPUs, one or more DSPs, one or more ASICs, or one or more FPGAs, as well as other discrete or integrated logic circuitry. The functions attributed to processing circuitry 50 herein may be embodied as software, firmware, hardware, or any combination thereof. In some examples, memory 52 includes computer-readable instructions that, when executed by processing circuitry 50, cause IMD 10 and processing circuitry 50 to perform various functions attributed herein to IMD 10 and processing circuitry 50. Memory 53 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital media.

Sensing circuitry 54 may monitor signals from electrodes 56 in order to, for example, monitor electrical activity of a heart of patient 4 and produce ECG data for patient 4. In some examples, processing circuitry 50 may identify features of the sensed ECG, such as heart rate, heart rate variability, intra-beat intervals, and/or ECG morphologic features, to detect an episode of cardiac arrhythmia of patient 4. Processing circuitry 50 may store the digitized ECG and features of the ECG used to detect the arrhythmia episode in memory 52 as episode data for the detected arrhythmia episode.

In some examples, sensing circuitry 54 measures impedance, e.g., of tissue proximate to IMD 10, via electrodes 56. The measured impedance may vary based on respiration and a degree of perfusion or edema. Processing circuitry 50 may determine physiological data relating to respiration, perfusion, and/or edema based on the measured signals such as impedance.

In some examples, sensing circuitry 54 measures an electromyogram signal, e.g., of tissue proximate to IMD 10, via electrodes 56. The measured electromyogram signal may vary based on respiration. Processing circuitry 50 may determine physiological data relating to respiration based on the measured electromyogram signal.

In some examples, sensing circuitry 54 measures a soundwave signal, e.g., produced by breathing sounds, via a microphone. The measured soundwave signal may vary based on respiration may be used to detect agonal breathing. Processing circuitry 50 may determine physiological data relating to respiration based on the measured soundwave signal.

In some examples, IMD 10 includes one or more sensors 58, such as one or more accelerometers, microphones, optical sensors, temperature sensors, and/or pressure sensors. In some examples, sensing circuitry 52 may include one or more filters and amplifiers for filtering and amplifying signals received from one or more of electrodes 56 and/or sensors 58. In some examples, sensing circuitry 54 and/or processing circuitry 50 may include a rectifier, filter and/or amplifier, a sense amplifier, comparator, and/or analog-to-digital converter. Processing circuitry 50 may determine physiological data, e.g., values of physiological parameters of patient 4, based on signals from sensors 58, which may be stored in memory 52.

Memory 52 may store applications executable by processing circuitry 50, and data 80. Applications may include an acute health event surveillance application. Processing circuitry 50 may execute event surveillance application to detect an acute health event of patient 4 based on combination of one or more of the types of physiological data described herein, which may be stored as sensed data. In some examples, sensed data may additionally include data sensed by other devices, e.g., computing device(s) 12, and received via communication circuitry 60. Event surveillance application may be configured with a rules engine that may include rules. The rules may include one or more models, algorithms, decision trees, and/or thresholds. In some cases, rules may be developed based on machine learning.

As examples, event surveillance application may detect a sudden cardiac arrest, a ventricular fibrillation, a ventricular tachycardia, a cardiac pause of asystole, pulseless electrical activity (PEA), or a myocardial infarction based on an ECG and/or other physiological data indicating the electrical or mechanical activity of heart 6 of patient 4 (FIG. 1). In some examples, event surveillance application may detect stroke based on such cardiac activity data. In some examples, sensing circuitry 54 may detect brain activity data, e.g., an electroencephalogram (EEG) via electrodes 56, and event surveillance application may detect stroke or a seizure based on the brain activity alone, or in combination with cardiac activity data or other physiological data. In some examples, event surveillance application detects whether the patient has fallen based on data from an accelerometer alone, or in combination with other physiological data. When event surveillance application detects an acute health event, event surveillance application may store the sensed data that lead to the detection (and in some cases a window of data preceding and/or following the detection) as event data.

In some examples, in response to detection of an acute health event, processing circuitry 50 transmits, via communication circuitry 60, event data for the event to computing device(s) 12 (FIG. 1). This transmission may be included in a message indicating the acute health event, as described herein. Transmission of the message may occur on an ad hoc basis and as quickly as possible. Communication circuitry 60 may include any suitable hardware, firmware, software, or any combination thereof for wirelessly communicating with another device, such as computing devices 12 and/or IoT devices 30.

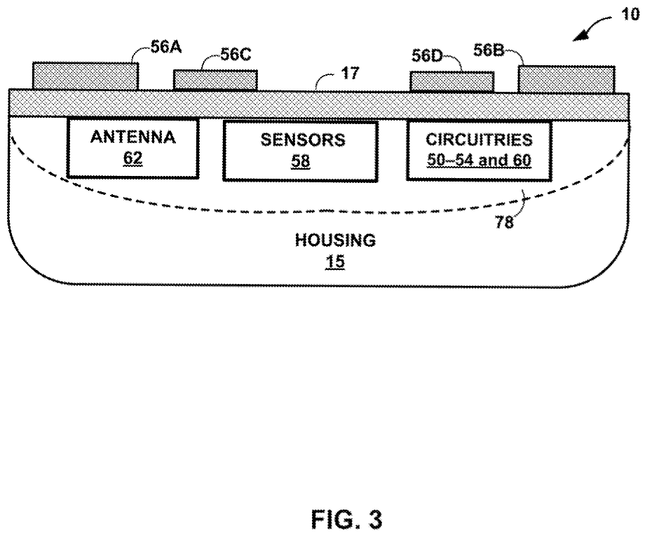

FIG. 3 is a conceptual drawing illustrating an example configuration of IMD 10 of FIGS. 1 and 2. In addition to the components illustrated in FIGS. 1-2, the example configuration of IMD 1A illustrated in FIG. 3 also may include a cover 17 and housing 15, which may help electrically insulate and protect circuitries 50-54 and 60, and sensors 58 therein. In some examples, insulative cover may be positioned over a housing 15 to form the housing for the components of IMD 10. One or more components of IMD 10 (e.g., antenna 62, sensors 58, processing circuitry 50, sensing circuitry 54, and communication circuitry 60) may be formed on a bottom side of the insulative cover. A power source for IMD 10 may be located within housing 15.

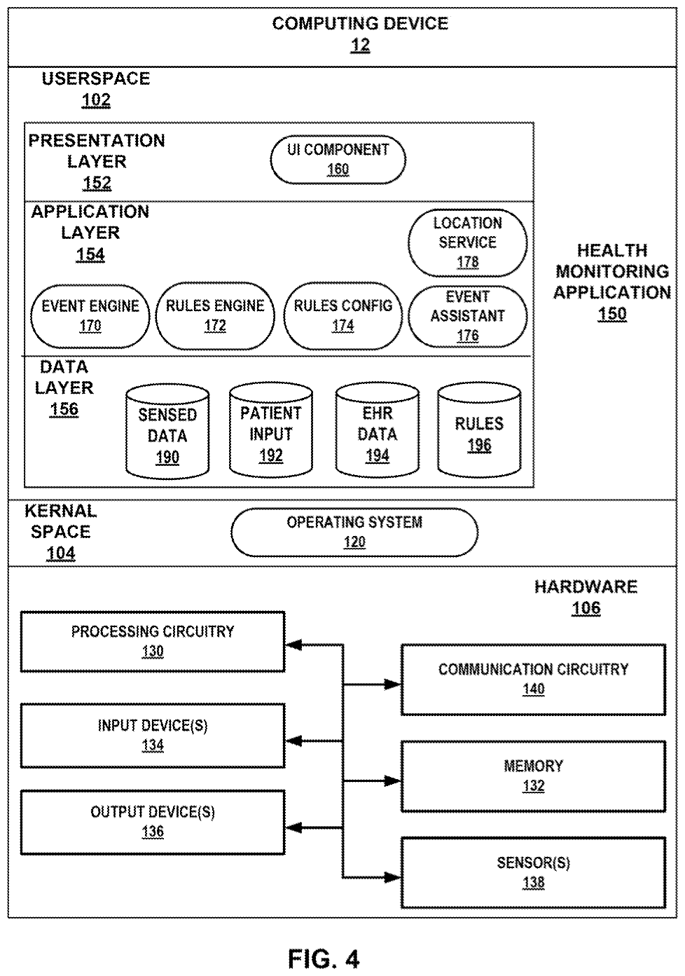

FIG. 4 is a block diagram illustrating an example configuration of a computing device 12 of patient 4, which may correspond to either (or both operating in coordination) of computing devices 12A and 12B illustrated in FIG. 1. In some examples, computing device 12 takes the form of a smartphone, a laptop, a tablet computer, a personal digital assistant (PDA), a smartwatch or other wearable computing device. In some examples, IoT devices 30 may be configured similarly to the configuration of computing device 12 illustrated in FIG. 4.

As shown in the example of FIG. 4, computing device 12 may be logically divided into user space 102, kernel space 104, and hardware 106. Hardware 106 may include one or more hardware components that provide an operating environment for components executing in user space 102 and kernel space 104. User space 102 and kernel space 104 may represent different sections or segmentations of memory, where kernel space 104 provides higher privileges to processes and threads than user space 102. For instance, kernel space 104 may include operating system 120, which operates with higher privileges than components executing in user space 102.

As shown in FIG. 4, hardware 106 includes processing circuitry 130, memory 132, one or more input devices 134, one or more output devices 136, one or more sensors 138, and communication circuitry 140. Although shown in FIG. 4 as a stand-alone device for purposes of example, computing device 12 may be any component or system that includes processing circuitry or other suitable computing environment for executing software instructions and, for example, need not necessarily include one or more elements shown in FIG. 4.

Processing circuitry 130 is configured to implement functionality and/or process instructions for execution within computing device 12. For example, processing circuitry 130 may be configured to receive and process instructions stored in memory 132 that provide functionality of components included in kernel space 104 and user space 102 to perform one or more operations in accordance with techniques of this disclosure. Examples of processing circuitry 130 may include, any one or more microprocessors, controllers, GPUs, TPUs, DSPs, ASICs, FPGAs, or equivalent discrete or integrated logic circuitry.

Memory 132 may be configured to store information within computing device 12, for processing during operation of computing device 12. Memory 132, in some examples, is described as a computer-readable storage medium. In some examples, memory 132 includes a temporary memory or a volatile memory. Examples of volatile memories include random access memories (RAM), dynamic random access memories (DRAM), static random access memories (SRAM), and other forms of volatile memories known in the art. Memory 132, in some examples, also includes one or more memories configured for long-term storage of information, e.g., including non-volatile storage elements. Examples of such non-volatile storage elements include magnetic hard discs, optical discs, floppy discs, flash memories, or forms of electrically programmable memories (EPROM) or electrically erasable and programmable (EEPROM) memories.

One or more input devices 134 of computing device 12 may receive input, e.g., from patient 4 or another user. Examples of input are tactile, audio, kinetic, and optical input. Input devices 134 may include, as examples, a mouse, keyboard, voice responsive system, camera, buttons, control pad, microphone, presence-sensitive or touch-sensitive component (e.g., screen), or any other device for detecting input from a user or a machine.

One or more output devices 136 of computing device 12 may generate output, e.g., to patient 4 or another user. Examples of output are tactile, audio, and visual output. Output devices 134 of computing device 12 may include a presence-sensitive screen, sound card, video graphics adapter card, speaker, cathode ray tube (CRT) monitor, liquid crystal display (LCD), light emitting diodes (LEDs), or any type of device for generating tactile, audio, and/or visual output.

One or more sensors 138 of computing device 12 may sense physiological parameters or signals of patient 4. Sensor(s) 138 may include electrodes, 3-axis accelerometers, an optical sensor, impedance sensors, temperature sensors, pressure sensors, heart sounds sensors, and other sensors, and sensing circuitry (e.g., including an ADC), similar to those described above with respect to IMD 10 and FIG. 2.

Communication circuitry 140 of computing device 12 may communicate with other devices by transmitting and receiving data. Communication circuitry 140 may include a network interface card, such as an Ethernet card, an optical transceiver, a radio frequency transceiver, or any other type of device that can send and receive information. For example, communication circuitry 140 may include a radio transceiver configured for communication according to standards or protocols, such as 3G, 4G, 5G, WiFi (e.g., 802.11 or 802.15 ZigBee), Bluetooth®, or Bluetooth® Low Energy (BLE).

As shown in FIG. 4, health monitoring application 150 executes in user space 102 of computing device 12. Health monitoring application 150 may be logically divided into presentation layer 152, application layer 154, and data layer 156. Presentation layer 152 may include a user interface (UI) component 160, which generates and renders user interfaces of health monitoring application 150.

Application layer 154 may include, but is not limited to, an event engine 170, rules engine 172, rules configuration component 174, event assistant 176, and location service 178. Event engine 172 may be responsive to receipt of an alert transmission from IMD 10 indicating that IMP 10 detected an acute health event. Event engine 172 may control performance of any of the operations in response to detection of an acute health event ascribed herein to computing device 12, such as activating an alarm, transmitting alert messages to HMS 22, controlling IoT devices 30, and analyzing data to confirm or override the detection of the acute health event by IMD 10.

Rules engine 174 analyzes sensed data 190, and in some examples, patient input 192 and/or EHR data 194, to determine whether there is a sufficient likelihood that patient 4 is experiencing the acute health event detected by ENID 10. Sensed data 190 may include data received from IMD 10 as part of the alert transmission, additional data transmitted from IMD 10, e.g., in “real-time,” and physiological and other data related to the condition of patient 4 collected by computing device(s) 12 and/or IoT devices 30. As examples sensed data 190 from computing device(s) 12 may include one or more of: activity levels, walking/running distance, resting energy, active energy, exercise minutes, quantifications of standing, body mass, body mass index, heart rate, low, high, and/or irregular heart rate events, heart rate variability, walking heart rate, heart beat series, digitized ECG, blood oxygen saturation, blood pressure (systolic and/or diastolic), respiratory rate, maximum volume of oxygen, blood glucose, peripheral perfusion, and sleep patterns.

Patient input 192 may include responses to queries posed by health monitoring application 150 regarding the condition of patient 4, input by patient 4 or another user, such as bystander 26. The queries and responses may occur responsive to the detection of the event by ID 10, or may have occurred prior to the detection, e.g., as part long-term monitoring of the health of patient 4. User recorded health data may include one or more of: exercise and activity data, sleep data, symptom data, medical history data, quality of life data, nutrition data, medication taking or compliance data, allergy data, demographic data, weight, and height. EHR data 194 may include any of the information regarding the historical condition or treatments of patient 4 described above. EHR data 194 may relate to history of cardiac arrest, tachyarrhythmia, myocardial infarction, stroke, seizure, chronic obstructive pulmonary disease (COPD), renal dysfunction, or hypertension, history of procedures, such as ablation or cardioversion, and healthcare utilization. EHR data 194 may also include demographic and other information of patient 4, such as age, gender, height, weight, and BMI.

Rules engine 172 may apply rules 196 to the data. Rules 196 may include one or more models, algorithms, decision trees, and/or thresholds. In some examples rules 196 may include hierarchical rules or thresholds that may be personalized to each patient based on their clinical history. In some cases, rules 196 may be developed based on machine learning. In some examples, rules 196 and the operation of rules engine 172 may provide a more complex analysis of the data. In some examples, rules 196 include one or more models developed by machine learning, and rules engine 172 applies feature vectors derived from the data to the model(s).

Rules configuration component 174 may be configured to modify rules 196 based on feedback indicating whether the detections and confirmations of acute health events by IMD 10 and computing device 12 were accurate. The feedback may be received from patient 4, or from care providers 40 and/or EHR 24 via HMS 22. In some examples, rules configuration component 174 may utilize the data sets from true and false detections and confirmations for supervised machine learning to further train models included as part of rules 196.

As discussed above, event assistant 176 may provide a conversational interface for patient 4 and/or bystander 26 to exchange information with computing device 12. Event assistant 176 may query the user regarding the condition of patient 4 in response to receiving the alert message from IMD 10. Responses from the user may be included as patient input 192. Event assistant 176 may use natural language processing and context data to interpret utterances by the user. In some examples, in addition to receiving responses to queries posed by the assistant, event assistant 176 may be configured to respond to queries posed by the user. In some examples, event assistant 176 may provide directions to and respond to queries regarding treatment of patient 4 from patient 4 or bystander 26.

Location service 178 may determine the location of computing device 12 and, thereby, the presumed location of patient 4. Location service 178 may use global position system (GPS) data, multilateration, and/or any other known techniques for locating computing devices.

In some examples, processing circuitry 130, e.g., implementing rules engine 172, may receive periodic respiratory parameter information, e.g., from IMD 10. Processing circuitry 130 may determine, e.g., by applying the periodic respiratory parameter information to rules 196, whether sudden cardiac arrest is detected.

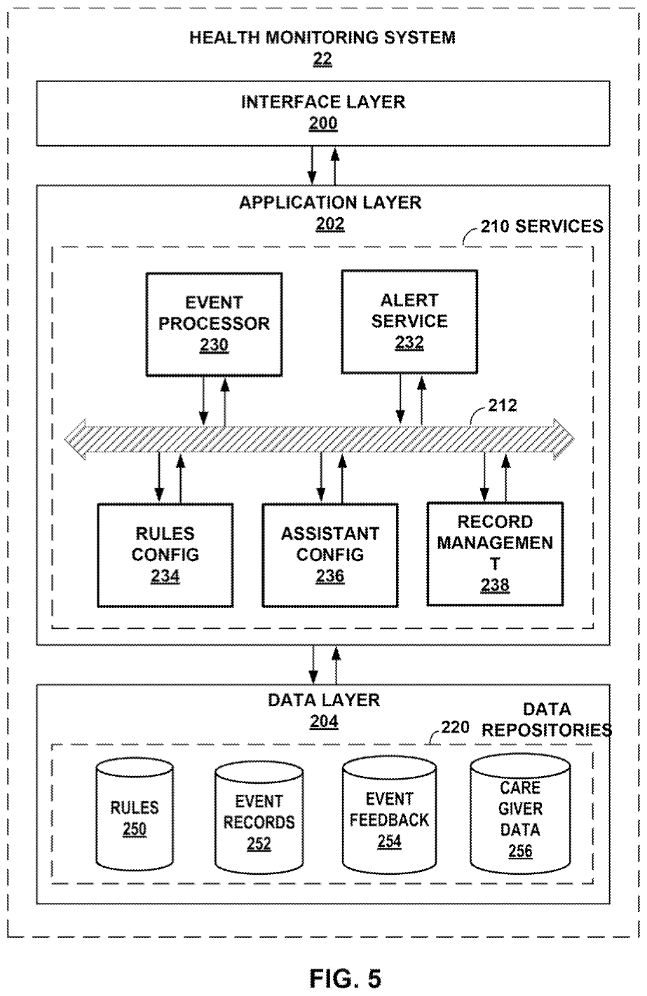

FIG. 5 is a block diagram illustrating an operating perspective of HMS 22. HMS 22 may be implemented in a computing system 20, which may include hardware components such as those of computing device 12, embodied in one or more physical devices. FIG. 5 provides an operating perspective of HMS 22 when hosted as a cloud-based platform. In the example of FIG. 5, components of HMS 22 are arranged according to multiple logical layers that implement the techniques of this disclosure. Each layer may be implemented by one or more modules comprised of hardware, software, or a combination of hardware and software.

Computing devices, such as computing devices 12, IoT devices 30, computing devices 38, and computing device 42, operate as clients that communicate with HMS 22 via interface layer 200. The computing devices typically execute client software applications, such as desktop application, mobile application, and web applications. Interface layer 200 represents a set of application programming interfaces (API) or protocol interfaces presented and supported by HMS 22 for the client software applications. Interface layer 200 may be implemented with one or more web servers.

As shown in FIG. 5, HMS 22 also includes an application layer 202 that represents a collection of services 210 for implementing the functionality ascribed to HMS herein. Application layer 202 receives information from client applications, e.g., an alert of an acute health event from a computing device 12 or IoT device 30, and further processes the information according to one or more of the services 210 to respond to the information. Application layer 202 may be implemented as one or more discrete software services 210 executing on one or more application servers, e.g., physical or virtual machines. That is, the application servers provide runtime environments for execution of services 210. In some examples, the functionality interface layer 200 as described above and the functionality of application layer 202 may be implemented at the same server. Services 210 may communicate via a logical service bus 212. Service bus 212 generally represents a logical interconnections or set of interfaces that allows different services 210 to send messages to other services, such as by a publish/subscription communication model.

Data layer 204 of HMS 22 provides persistence for information in PPEMS 6 using one or more data repositories 220. A data repository 220, generally, may be any data structure or software that stores and/or manages data. Examples of data repositories 220 include but are not limited to relational databases, multi-dimensional databases, maps, and hash tables, to name only a few examples.

As shown in FIG. 5, each of services 230-238 is implemented in a modular form within HMS 22. Although shown as separate modules for each service, in some examples the functionality of two or more services may be combined into a single module or component. Each of services 230-238 may be implemented in software, hardware, or a combination of hardware and software. Moreover, services 230-238 may be implemented as standalone devices, separate virtual machines or containers, processes, threads or software instructions generally for execution on one or more physical processors.

Event processor service 230 may be responsive to receipt of an alert transmission from computing device(s) 12 and/or IoT device(s) 30 indicating that IMD 10 detected an acute health event of patient and, in some examples, that the transmitting device confirmed the detection. Event processor service 230 may initiate performance of any of the operations in response to detection of an acute health event ascribed herein to HMS 22, such as communicating with patient 4, bystander 26, and care providers 40, activating drone 46 and, in some cases, analyzing data to confirm or override the detection of the acute health event by IMD 10.

Record management service 238 may store the patient data included in a received alert message within event records 252. Alert service 232 may package the some or all of the data from the event record, in some cases with additional information as described herein, into one more alert messages for transmission to bystander 26 and/or care providers 40. Care giver data 256 may store data used by alert service 232 to identify to whom to send alerts based on locations of potential bystanders 26 and care givers 40 relative to a location of patient 4 and/or applicability of the care provided by care givers 40 to the acute health event experienced by patient 4.

In examples in which HMS 22 performs an analysis to confirm or override the detection of the acute health event by IMD 10, event processor service 230 may apply one or more rules 250 to the data received in the alert message, e.g., to feature vectors derived by event processor service 230 from the data. Rules 250 may include one or more models, algorithms, decision trees, and/or thresholds, which may be developed by rules configuration service 234 based on machine learning. Example machine learning techniques that may be employed to generate rules 250 can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

In some examples, in addition to rules used by HMS 22 to confirm acute health event detection, (or in examples in which HMS 22 does not confirm event detection) rules 250 maintained by HMS 22 may include rules 196 utilized by computing devices 12 and rules used by IMD 10. In such examples, rules configuration service 250 may be configured to develop and maintain rules 196. Rules configuration service 234 may be configured to modify these rules based on event feedback data 254 that indicates whether the detections and confirmations of acute health events by IMD 10, computing device 12, and/or HMS 22 were accurate. Event feedback 254 may be received from patient 4, e.g., via computing device(s) 12, or from care providers 40 and/or EHR 24. In some examples, rules configuration service 234 may utilize event records from true and false detections (as indicated by event feedback data 254) and confirmations for supervised machine learning to further train models included as part of rules 250.

As illustrated in the example of FIG. 5, services 210 may also include an assistant configuration service 236 for configuring and interacting with event assistant 176 implemented in computing device 12 or other computing devices.

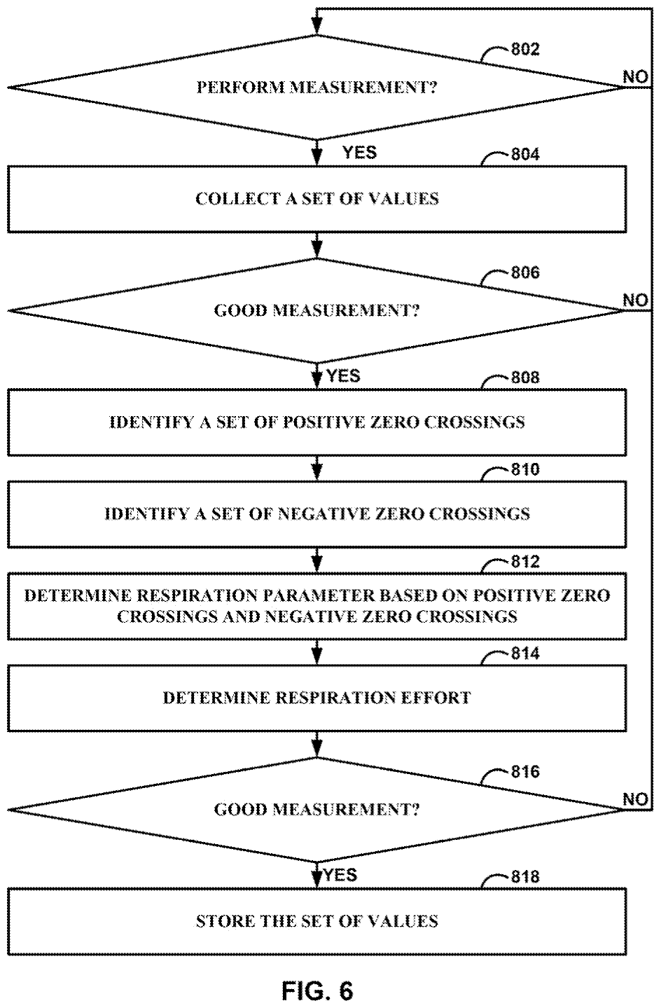

FIG. 6 is a flow diagram illustrating an example operation for performing respiration parameter measurements, in accordance with one or more techniques of this disclosure. For convenience, FIG. 6 is described with respect to IMD 10 and computing device(s) 12 of FIG. 1. However, the techniques of FIG. 6 may be additionally or alternatively performed by different components of system 2.

For example, IMD 10 may perform a set of measurements, where each measurement produces data consisting of a set of values. In some cases, the data may be indicative of one or more physiological functions of patient 4, such as any combination of cardiac functions, respiratory functions, and intestinal functions. For example, processing circuitry 50 may process the data to determine one or more parameters (e.g., respiratory rate, respiratory rate variability, and respiratory effort) or respiratory parameter information associated with respiratory cycles of the patient. In turn, processing circuitry 50 may analyze such parameters to identify or monitor one or more medical conditions. Since it may be beneficial to track respiratory parameters over an extended period of time, IMD 10 may, in some examples, perform measurements at a measurement rate, as described in further detail below.

In some examples, IMD 10 performs measurements according to a measurement schedule that is uploaded to IMD 10 by computing device(s) 12 or another device, e.g., computing system 20A via computing device(s) 12. In one or more examples, the measurements are performed continuously. In one or more examples, the measurements are performed every ⅛ second, every ½ second, every second, every other second, or every minute. In some examples, measurements will occur when triggered by a sensor detecting a signal indicating a change from a baseline signal. In some examples, measurements will be triggered when a change from the baseline signal indicates a worsening condition.

The measurement schedule may, in some cases, be stored in memory 52 of IMD 10. In some examples, the measurement schedule may include an instruction to perform measurements at a measurement rate (e.g., one measurement per hour, one measurement per day, one measurement per month, or any other valid rate). Additionally, in some examples, the measurement schedule includes instructions to perform measurements based on a time of day. For example, the measurement may include instructions to perform measurements only at daytime (e.g., from 8 AM to 8 PM), only at nighttime (e.g., from 12 AM to 6 AM), or instructions to perform measurements at a first measurement rate during the daytime and perform measurements at a second measurement rate during the nighttime, where the first measurement rate is different from the second measurement rate. Each measurement may last for a measurement duration, where the measurement duration may be set based on instructions received by IMD 10 from external device 12 or another device. In some examples, the measurement duration is within a range between 10 seconds and 60 seconds (e.g., 32 seconds). In this way, each measurement may capture a plurality of respiratory cycles, where each respiratory cycle includes an inhale phase and an exhale phase. In some examples, the measurement duration may be 10 seconds. In some examples, the measurement duration is within a range between 3 seconds and 4 seconds. In some examples, the measurement duration may be for two or more respiratory cycles.

Additionally, since it may be beneficial to perform measurements under certain conditions, IMD 10 may, in some examples, perform measurements in response to a set of patient parameters or based on detection of an event, as described in further detail below. In some examples, the set of patient parameters includes any combination of a heart rate of patient 4, a posture of patient 4, an activity level of patient 4, an electrocardiogram (ECG) corresponding to patient 4, a presence or an absence of one or more arrhythmias, patient triggers, and data from an acoustic sensor. In some examples, detection of an event may include detection of an SCA. IMD 10 may measure each parameter of the set of patient parameters at a respective parameter measurement rate. The parameter measurement rate corresponding to each patient parameter of the set of patient parameters may, in some cases, be stored in memory 52 of IMD 10.

At block 802, IMD 10 may determine whether to perform a measurement. In some examples, IMD 10 determines whether to perform the measurement based on the measurement schedule which is stored in memory 52. Additionally, in some examples, IMD 10 determines whether to perform the measurement based on a set of patient parameters. If IMD 10 determines not to perform a measurement (“NO” branch of block 802), IMD 10 continues to determine whether to perform a measurement. If IMD 10 determines to perform an evaluation (“YES” branch of block 802), IMD 10 proceeds to collect a set of values (804). In some examples, IMD 10 collects the set of values at a sampling rate between 100 Hz and 300 Hz. In some cases, the sampling rate is 128 Hz. In other cases, the sampling rate is 256 Hz. In some examples, each value of the set of values defines a resolution between 10 bits and 20 bits (e.g., 14 bits). IMD 10 may, in some cases truncate each value from a first resolution to a second resolution (e.g., from 14 bits to 12 bits).

After IMD 10 collects the set of values, processing circuitry 50 may determine, based on the set of values, whether the measurement is a good measurement (806). For example, if the accelerometer shows the patient is active, then the signal may not be good enough to estimate respiration rate and/or effort. Processing circuitry 50 may determine whether the measurement is a good measurement by comparing a maximum value of the set of values, a minimum value of the set of values, a difference between the maximum value and the minimum value, or a mean value of the set of values with a respective threshold. For example, if a difference between the maximum value and the minimum value is greater than a respective threshold (e.g., a “noise” threshold), then processing circuitry 50 may determine that a quality of the measurement is not sufficient to continue processing the set of values. If processing circuitry 50 determines that the measurement is not a good measurement (“NO” branch of block 806), the example operation may return to block 802. If processing circuitry 50 determines that the measurement is a good measurement (“YES” branch of block 806), the example operation may proceed to block 808.

Processing circuitry 50 may be configured to identify a set of positive zero crossings (808) and identify a set of negative zero crossings (810) in the set of values. In some examples, processing circuitry 50 may be configured to identify a set of non-zero threshold crossings. In some examples, processing circuitry 50 may process the set of values to filter out high-frequency and low-frequency components, and center the set of values around a y=0 axis. For example, the processed values may represent a signal that oscillates about the y=0 axis as patient 4 inhales and exhales. As such, the processed values may periodically transition from a negative value to a positive value (e.g., a negative-going-positive occurrence) or transition from a positive value to a negative value (e.g., a positive-going-negative occurrence). To determine the set of positive zero crossings and the set of negative zero crossings, in some cases, processing circuitry 50 may determine whether each negative-going-positive occurrence satisfies a first set of conditions and if each positive-going-negative occurrence satisfies a second set of conditions. Processing circuitry 50 may determine that negative-going-positive occurrences that satisfy the first set of conditions represent the set of positive zero crossings and that positive-going-negative occurrences that satisfy the second set of conditions represent the set of negative zero crossings.

Processing circuitry 50 determines one or more respiration parameters based on the set of positive zero crossings and the set of negative zero crossings (812). For example, processing circuitry 50 may determine a respiration rate and a respiration rate variability using the set of positive zero crossings and the set of negative zero crossings. Since the processed set of values may represent an oscillating signal indicative of respiratory patterns of patient 4, a single respiration cycle may be given by an amount of time separating consecutive positive zero crossings of the set of positive zero crossings or an amount of time separating consecutive negative zero crossings of the set of negative zero crossings. As such, processing circuitry 50 may determine a set of respiration cycles, or respiration intervals based on the set of positive zero crossings and the set of negative zero crossings. Using the set of respiration cycles, processing circuitry 50 may calculate a mean respiration cycle, determine a median respiration cycle, calculate a variability in the set of respiration, or any combination thereof. To calculate a respiration rate corresponding to the set of values collected during a respective measurement, for example, processing circuitry 50 may calculate a respiration rate corresponding to a median respiration cycle of the set of respiration cycles.

Processing circuitry 50 determines a respiration effort (814) based on, in some examples, the set of positive zero crossings, the set of negative zero crossings, and the set of values. Respiration effort may, at least in part, be given by an amplitude of the signal represented by the set of values. In other words, deeper breathing may result in greater signal amplitudes than shallower breathing. In some cases, processing circuitry 50 may determine a single respiration effort value corresponding to the set of values.

At block 816, processing circuitry 50 may determine whether the measurement producing the set of values is a good measurement. To determine whether the measurement is a good measurement, processing circuitry 50 may compare a set of parameter values (e.g., a motion level associated with patient 4, respiration effort, heart rate, heart rate variability, ambient light, or any combination thereof) associated with the measurement with respective threshold parameter values. Additionally, or alternatively, in some cases, processing circuitry 50 may determine whether the parameter measurement satisfies a set of conditions. If processing circuitry 50 determines that the measurement is not a good measurement (“NO” branch of block 816), the operation returns to block 802 and IMD 10 determines whether to perform another measurement. If processing circuitry 50 determines that the measurement is a good measurement (“YES” branch of block 816), processing circuitry 50 stores the set of values (818). In some examples, processing circuitry 50 stores the set of values in memory 52 of IMD 10, storage device 84 of external device 12, or another storage device not pictured in FIGS. 1-2. Additionally, in some examples, processing circuitry 50 may store the set of respiration intervals (e.g., a group of respiration intervals derived from positive zero crossings (Pi) and a group of respiration intervals derived from negative zero crossings (Ni).

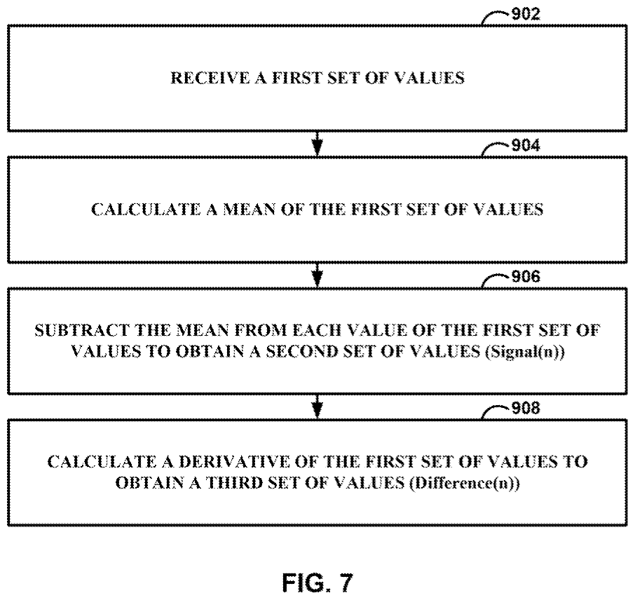

FIGS. 7-9 illustrate example operations for identifying a set of positive zero crossings and a set of negative zero crossings based on a set of values, by first processing the set of values.

FIG. 7 is a flow diagram illustrating an example operation for processing a set of values, in accordance with one or more techniques of this disclosure. For convenience, FIG. 7 is described with respect to IMD 10, computing device(s) 12, and processing circuitry 50 of FIGS. 1-2. However, the techniques of FIG. 7 may additionally or alternatively be performed by different components of IMD 10, computing device 12, processing circuitry 50, system 2, or by additional or alternative medical devices.

When IMD 10 performs a measurement, IMD may collect a set of values which represent a signal over a period of time. The set of values collected by IMD 10 may be referred to as the “raw signal.” The signal may be analyzed by processing circuitry 50 in order to determine respiratory parameters, such as respiratory rate, respiratory rate variability, and respiratory effort, as examples. In some examples, it may be beneficial for processing circuitry 50 to process the raw signal before analyzing the values to determine respiratory parameters.

As illustrated in FIG. 7, processing circuitry 50 receives a first set of values (902). Processing circuitry 50 may, in some cases, represent processing circuitry 50 of IMD 10. In such cases, processing circuitry 50 may access memory 52 to obtain the first set of values. Additionally, in some cases, processing circuitry 50 represents processing circuitry 80 of external device 12. In such cases, processing circuitry 50 may access memory 52, or access another storage device to obtain the first set of values. The first set of values may represent data corresponding to a measurement. Additionally, in some cases, the first set of values may include a timestamp which indicates a time in which the respective measurement was taken. In some examples, each pair of consecutive values of the first set of values is separated by a sampling interval. In other words, the first set of values may define a sampling rate. The sampling rate may be within a range between 5 Hz and 16 Hz (e.g., 8 Hz) and a duration of the first set of values may be within a range between 10 seconds and 60 seconds (e.g., 32 seconds).

Processing circuitry 50 calculates a mean value corresponding to each value of the first set of values (904) and subtracts the respective mean value from each value of the first set of values to obtain a second set of values (906). In some examples, the mean value corresponding to each value of the first set of values represents a mean value of the first set of values. In such examples, the second set of values may resemble the first set of values when the sets are plotted, with the second set of values being offset by the mean value on the y-axis. Such an offset may center the second set of values about a y=0 axis. Consequently, since the data may, in some cases, show an oscillation representative of patient 4 inhaling and exhaling, the second set of values may oscillate about the y=0 axis. In some examples, the mean value corresponding to each value of the first set of values represents a moving average of the first set of values. For example, processing circuitry 50 may apply an m-sample moving average, where the respective mean value corresponding to each value represents a mean value of m (e.g., m=64 when first set of values is sampled at 8 Hz) values preceding the respective value. Additionally, in some cases, processing circuitry 50 may apply a high pass filter to the first set of values in order to obtain the second set of values. In the example operation of FIG. 7, the second set of values is represented by Signal(n), where the second set of values is n values in length.

After obtaining the second set of values, processing circuitry 50 calculates a derivative of the first set of values to obtain a third set of values (908). For example, to calculate the derivative, processing circuitry 50 may determine a difference value associated with each value of the first set of values. Each difference value may represent a difference between a first value preceding the respective value and a second value following the respective value. In some examples, processing circuitry 50 sets the first three values of the third set of values to zero. In the example operation of FIG. 7, the third set of values is represented by Difference(n), where the third set of values is n values in length.

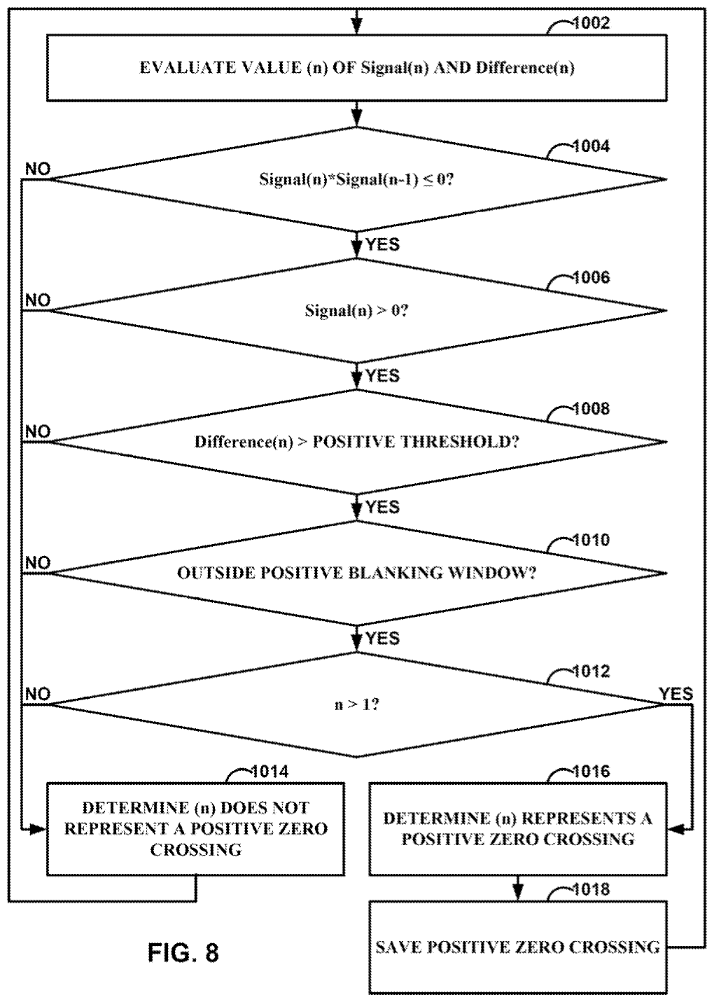

FIG. 8 is a flow diagram illustrating an example operation for determining a set of positive zero crossings in a measurement, in accordance with one or more techniques of this disclosure. For convenience, FIG. 8 is described with respect to IMD 10, external device 12, and processing circuitry 50 of FIGS. 1-2. However, the techniques of FIG. 8 may be performed by different components of IMD 10, external device 12, processing circuitry 50, or by additional or alternative medical devices.

In some examples, processing circuitry 50 determines one or more respiration parameters based, at least in part, on a set of positive zero crossings. For example, processing circuitry 50 may determine a set of respiration intervals based on the set of positive zero crossings. Processing circuitry 50 may determine the set of positive zero crossings in data collected by IMD 10, where the data is collected during a measurement. ID 10 may, in some examples, be an ICM implanted in patient 4 that measures patient parameters for analysis to identify or monitor one or more patient conditions such as SCA. For example, IMD 10 may perform a set of measurements, where each measurement produces data consisting of a set of values. In some cases, the data may be indicative of one or more physiological functions of patient 4, such as any combination of cardiac functions, respiratory functions, and intestinal functions. For example, processing circuitry 50 may process the data to determine one or more parameters (e.g., respiratory rate, respiratory rate variability, and respiratory effort) associated with respiratory cycles of the patient, as discussed in further detail below. In turn, processing circuitry 50 may analyze such parameters to identify or monitor one or more medical conditions. To determine the set of positive zero crossings, processing circuitry 50 may determine whether each positive-going-positive occurrence in the second set of values (Signal(n)) satisfies a set of conditions. In the example operation of FIG. 8, the set of conditions may include the decisions of blocks 1004-1012, as described in further detail below.

As illustrated in FIG. 8, processing circuitry 50 may evaluate a value n of the second set of values (e.g., Signal(n)) and a corresponding value of the third set of values (e.g., Difference(n)) (1002) to determine if n represents a positive zero crossing (e.g., a positive zero crossing 712 as illustrated in FIG. 7). In some examples, the second set of values and the first set of values may be calculated based on a first set of values collected by IMD 10 (e.g., raw data).

At block 1004, processing circuitry 50 may determine, for each value of the second set of values, whether a multiplication of two consecutive values of the second set of values (e.g., Signal(n)·Signal(n−1)) is less than or equal to zero. In some examples, if the multiplication of two consecutive values of the second set of values is less than or equal to zero, this may indicate that one of Signal(n) and Signal(n−1) is negative and one of Signal(n) and Signal(n−1) is positive. Additionally, in some examples, if the multiplication of two consecutive values of the second set of values is less than or equal to zero, this may indicate that at least one of Signal(n) and impedanceSignal(n−1) is equal to zero. As such, by multiplying two consecutive values, processing circuitry 50 may determine whether the respective consecutive values represent a zero crossing event. If the multiplication of two consecutive values of the second set of values is not less than or equal to zero (“NO” branch of block 1004), processing circuitry 50 determines that the respective consecutive values do not satisfy the condition of block 1004, and processing circuitry 50 determines that Signal(n) does not represent a positive zero crossing (1014). If the multiplication of two consecutive values of the second set of values is less than or equal to zero (“YES” branch of block 1004), processing circuitry 50 determines that the respective consecutive values satisfy the condition of block 1004, and processing circuitry 50 proceeds to evaluate the condition of block 1006. Additionally, or alternatively, in some cases, processing circuitry may monitor impedanceSignal for sign changes at block 1004. If a sign change is detected, the condition of block 1004 is satisfied and if a sign change is not detected, the condition of block 1004 is not satisfied.

At block 1006, processing circuitry 50 may determine whether a value (e.g., Signal(n)) of a pair of consecutive values is greater than zero, where the pair of consecutive values (e.g., Signal(n) and Signal(n−1)) represent a zero crossing event identified by processing circuitry 50 at block 1004. If processing circuitry 50 determines that the value is not greater than zero (“NO” branch of block 1006), processing circuitry 50 determines that the respective consecutive values do not satisfy the condition of block 1006, and processing circuitry 50 determines that Signal(n) does not represent a positive zero crossing (1014). If processing circuitry 50 determines that the value is greater than zero (“YES” branch of block 1006), processing circuitry 50 determines that the value satisfies the condition of block 1006 and processing circuitry 50 proceeds to evaluate the condition of block 1008.

At block 1008, processing circuitry 50 may determine, if a difference value of the third set of values (e.g., Difference(n)) is greater than a positive threshold difference value (e.g., POSITIVE THRESHOLD). The difference value may, in some cases, represent a slope associated with a value (e.g., Signal(n)) of a pair of consecutive values that processing circuitry 50 determines to be a zero crossing event in block 1004. If processing circuitry 50 determines that the difference value is not greater than the positive threshold difference value (“NO” branch of block 1008), processing circuitry 50 determines that the respective consecutive values do not satisfy the condition of block 1008, and processing circuitry 50 determines that Signal(n) does not represent a positive zero crossing (1014). If processing circuitry 50 determines that the difference value is greater than the positive threshold difference value (“YES” branch of block 1008), processing circuitry 50 determines that the value satisfies the condition of block 1008 and processing circuitry 50 proceeds to evaluate the condition of block 1010.

At block 1010, processing circuitry 50 may determine whether the value (e.g., Signal(n)) of the respective pair of consecutive values that satisfies the conditions of blocks 1004-1008 is outside of a positive blanking window. To determine whether the value is outside of the positive blanking window, processing circuitry 50 determines whether the value is outside of a group of consecutive secondary values immediately preceding the secondary value n, where the group of consecutive secondary values represents the positive blanking window. In some examples, the group of consecutive secondary values includes a number of consecutive secondary values within a range between 5 and 15 (e.g., 10). If a positive zero crossing exists within the group of consecutive secondary values, processing circuitry 50 may determine that Signal(n) is not outside of the positive blanking window (“NO” branch of block 1010), determine that the condition of block 1010 is not satisfied for the secondary value n, and determine that that Signal(n) does not represent a positive zero crossing (1014). If a positive zero crossing does not exist within the group of consecutive secondary values, processing circuitry 50 may determine that Signal(n) is outside of the positive blanking window (“YES” branch of block 1010), determine that the condition of block 1010 is satisfied for the secondary value n, and proceed to evaluate the condition of block 1012. In this way, a positive blanking window may follow each valid positive zero crossing. If a potential positive zero crossing occurs within a blanking window following a valid positive zero crossing, processing circuitry 50 may determine that the potential positive zero crossing is invalid (e.g., does not represent a valid positive zero crossing).

At block 1012, processing circuitry 50 may determine whether n is greater than one. If n is not greater than 1, processing circuitry 50 may determine that the condition of block 1012 is not satisfied and determine that Signal(n) does not represent a positive zero crossing (1014). If n is greater than 1, processing circuitry 50 may determine that the condition of blocks 1004-1012 are satisfied and determine that Signal(n) represents a positive zero crossing (1016). Subsequently, processing circuitry 50 may save the positive zero crossing (1018) in a storage device (e.g., memory 52, storage device 84, or another storage device) as a part of the set of positive zero crossings.

Processing circuitry 50 may, in some examples, evaluate every pair of consecutive values of the second set of values to determine whether each respective pair of consecutive values represents a positive zero crossing. The conditions of blocks 1004-1012 may, in some cases be evaluated in a different order than illustrated in FIG. 8.

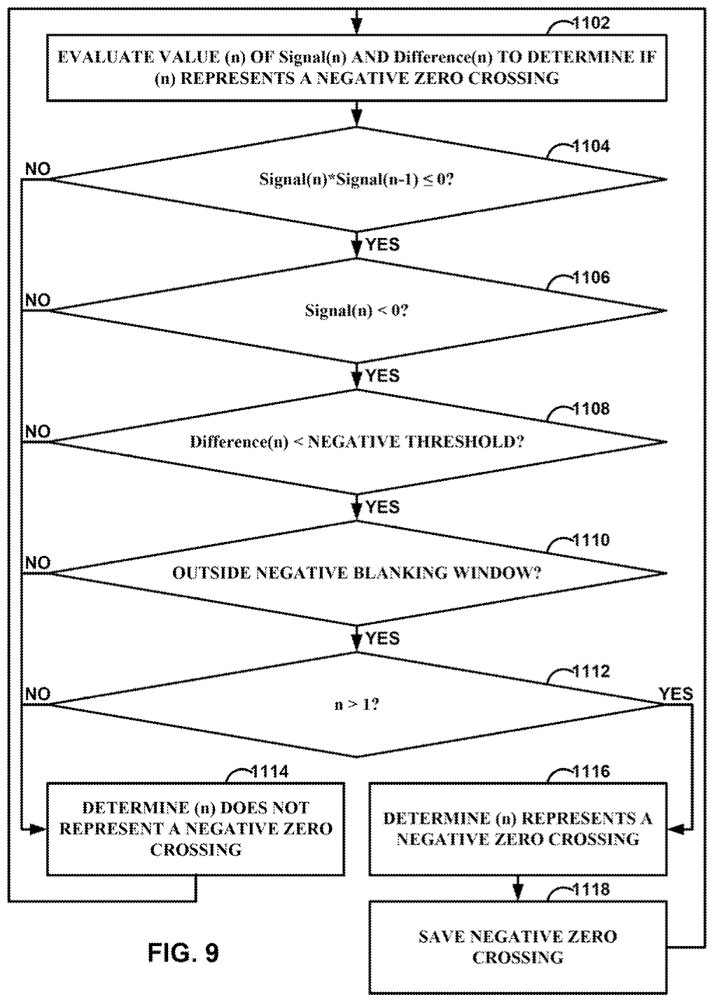

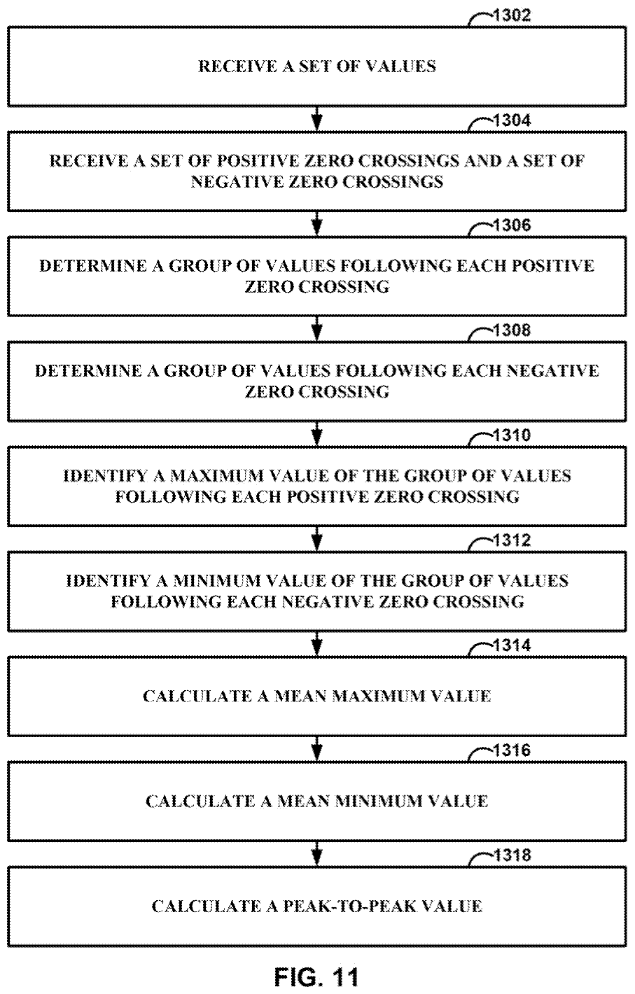

FIG. 9 is a flow diagram illustrating an example operation for determining a set of negative zero crossings in a measurement, in accordance with one or more techniques of this disclosure. For convenience, FIG. 8 is described with respect to IMD 10, external device 12, and processing circuitry 50 of FIGS. 1-2. However, the techniques of FIG. 11 may be performed by different components of IMD 10, computing device(s) 12, processing circuitry 50, system 2, or by additional or alternative medical devices.

In some examples, processing circuitry 50 determines one or more respiration parameters based, at least in part, on a set of negative zero crossings. For example, processing circuitry 50 may determine a set of respiration intervals based on the set of negative zero crossings. Processing circuitry 50 may determine the set of negative zero crossings in data collected by IMD 10, where the data is collected during a measurement. IMD 10 may, in some examples, be an ICM implanted in patient 4 that measures patient parameters for analysis to identify or monitor one or more patient conditions such as SCA, heart failure, sleep apnea, or COPD. For example, IMD 10 may perform a set of measurements, where each measurement produces data consisting of a set of values. In some cases, the data may be indicative of one or more physiological functions of patient 4, such as any combination of cardiac functions, and respiratory functions. For example, processing circuitry 50 may process the data to determine one or more parameters (e.g., respiratory rate, respiratory rate variability, and respiratory effort) associated with respiratory cycles of the patient, as discussed in further detail below. In turn, processing circuitry 50 may analyze such parameters to identify or monitor one or more medical conditions.

As illustrated in FIG. 9, processing circuitry 50 may evaluate a value n of the second set of values (e.g., Signal(n)) and a corresponding value of the third set of values (e.g., Difference(n)) (1102) to determine if n represents a negative zero crossing (e.g., a negative zero crossing 714 as illustrated in FIG. 7). In some examples, the second set of values and the first set of values may be calculated based on a first set of values collected by IMD 10 (e.g., raw data).

At block 1104, processing circuitry 50 may determine, for each value of the second set of values, whether a multiplication of two consecutive values of the second set of values (e.g., Signal(n)·Signal(n−1)) is less than or equal to zero. In some examples, if the multiplication of two consecutive values of the second set of values is less than or equal to zero, this may indicate that one of Signal(n) and Signal(n−1) is negative and one of Signal(n) and Signal(n−1) is positive. Additionally, in some examples, if the multiplication of two consecutive values of the second set of values is less than or equal to zero, this may indicate that at least one of Signal(n) and Signal(n−1) is equal to zero. As such, by multiplying two consecutive values, processing circuitry 50 may determine whether the respective consecutive values represent a zero crossing event. If the multiplication of two consecutive values of the second set of values is not less than or equal to zero (“NO” branch of block 1104), processing circuitry 50 determines that the respective consecutive values do not satisfy the condition of block 1104, and processing circuitry 50 determines that Signal(n) does not represent a negative zero crossing (1114). If the multiplication of two consecutive values of the second set of values is less than or equal to zero (“YES” branch of block 1104), processing circuitry 50 determines that the respective consecutive values satisfy the condition of block 1104, and processing circuitry 50 proceeds to evaluate the condition of block 1106. Additionally, or alternatively, in some cases, processing circuitry may monitor impedanceSignal for sign changes at block 1104. If a sign change is detected, the condition of block 1104 is satisfied and if a sign change is not detected, the condition of block 1104 is not satisfied.

At block 1106, processing circuitry 50 may determine whether a value (e.g., Signal(n)) of a pair of consecutive values is less than zero, where the pair of consecutive values (e.g., Signal(n) and Signal(n−1)) represent a zero crossing event identified by processing circuitry 50 at block 1104. If processing circuitry 50 determines that the value is not less than zero (“NO” branch of block 1106), processing circuitry 50 determines that the respective consecutive values do not satisfy the condition of block 1106, and processing circuitry 50 determines that Signal(n) does not represent a negative zero crossing (1114). If processing circuitry 50 determines that the value is less than zero (“YES” branch of block 1106), processing circuitry 50 determines that the value satisfies the condition of block 1106 and processing circuitry 50 proceeds to evaluate the condition of block 1108.

At block 1108, processing circuitry 50 may determine, if a difference value of the third set of values (e.g., Difference(n)) is less than a negative threshold difference value (e.g., NEGATIVE THRESHOLD). The difference value may, in some cases, represent a slope associated with a value (e.g., Signal(n)) of a pair of consecutive values that processing circuitry 50 determines to be a zero crossing event in block 1104. If processing circuitry 50 determines that the difference value is not less than the negative threshold difference value (“NO” branch of block 1108), processing circuitry 50 determines that the respective consecutive values do not satisfy the condition of block 1108, and processing circuitry 50 determines that Signal(n) does not represent a negative zero crossing (1114). If processing circuitry 50 determines that the difference value is less than the negative threshold difference value (“YES” branch of block 1108), processing circuitry 50 determines that the value satisfies the condition of block 1108 and processing circuitry 50 proceeds to evaluate the condition of block 1110.

At block 1110, processing circuitry 50 may determine whether the value (e.g., Signal(n)) of the respective pair of consecutive values that satisfies the conditions of blocks 1104-1108 is outside of a negative blanking window. To determine whether the value is outside of the negative blanking window, processing circuitry 50 determines whether the value is outside of a group of consecutive secondary values immediately preceding the secondary value n, where the group of consecutive secondary values represents the negative blanking window. In some examples, the group of consecutive secondary values includes a number of consecutive secondary values within a range between 5 and 15 (e.g., 10). If a negative zero crossing exists within the group of consecutive secondary values, processing circuitry 50 may determine that Signal(n) is not outside of the negative blanking window (“NO” branch of block 1110), determine that the condition of block 1110 is not satisfied for the secondary value n, and determine that that Signal(n) does not represent a negative zero crossing (1114). If a negative zero crossing does not exist within the group of consecutive secondary values, processing circuitry 50 may determine that Signal(n) is outside of the negative blanking window (“YES” branch of block 1110), determine that the condition of block 1110 is satisfied for the secondary value n, and proceed to evaluate the condition of block 1112. In this way, a negative blanking window may follow each valid negative zero crossing. If a potential negative zero crossing occurs within a blanking window following a valid negative zero crossing, processing circuitry 50 may determine that the potential negative zero crossing is invalid (e.g., does not represent a valid negative zero crossing).

At block 1112, processing circuitry 50 may determine whether n is greater than one. If n is not greater than 1, processing circuitry 50 may determine that the condition of block 1112 is not satisfied and determine that Signal(n) does not represent a negative zero crossing (1114). If n is greater than 1, processing circuitry 50 may determine that the condition of blocks 1104-1112 are satisfied and determine that Signal(n) represents a negative zero crossing (1116). Subsequently, processing circuitry 50 may save the negative zero crossing (1118) in a storage device (e.g., memory 52, or another storage device) as a part of the set of negative zero crossings.

Processing circuitry 50 may, in some examples, evaluate every pair of consecutive values of the second set of values to determine whether each respective pair of consecutive values represents a negative zero crossing. The conditions of blocks 1104-1112 may, in some cases be evaluated in a different order than illustrated in FIG. 9.

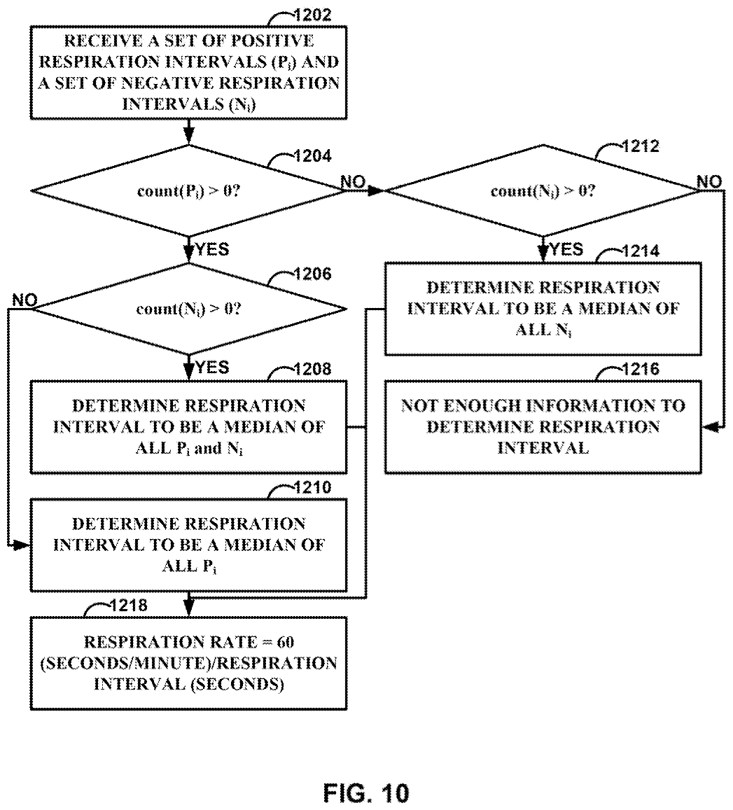

FIG. 10 is a flow diagram illustrating an example operation for determining a set of respiration intervals, in accordance with one or more techniques of this disclosure. For convenience, FIG. 10 is described with respect to IMD 10, computing device 12, and processing circuitry 50 of FIGS. 1-2. However, the techniques of FIG. 10 may be performed by different components of IMD 10, external device 12, processing circuitry 50, system 2, or by additional or alternative medical devices.

Processing circuitry 50 may determine, or in some cases receive, a set of respiration intervals derived from positive zero crossings (Pᵢ) and a set of respiration intervals derived from negative zero crossings (Nᵢ) (1202). The set of respiration intervals (Pi) may be determined based on a set of positive zero crossings and the set of respiration intervals (Ni) may be determined based on a set of negative zero crossings. Subsequently, processing circuitry 50 may determine if a number of respiration intervals of the set of respiration intervals (Pᵢ) is greater than zero (1204). If the number of respiration intervals (Pᵢ) is greater than zero (“YES” branch of block 1204), processing circuitry 50 determines if a number of respiration intervals of the set of respiration intervals (Nᵢ) is greater than zero (1206). Additionally, if the number of respiration intervals (Pᵢ) is not greater than zero (“NO” branch of block 1204), processing circuitry 50 determines if a number of respiration intervals of the set of respiration intervals (Nᵢ) is greater than zero (1212).

If, at block 1206, processing circuitry 50 determines that the number of respiration intervals (Nᵢ) is greater than zero (“YES” branch of block 1206), processing circuitry 50 may determine a respiration interval to be a median of all respiration intervals of the set of respiration intervals (Pᵢ) and all respiration intervals of the set of respiration intervals (Nᵢ) (1208). Additionally, in some cases where the number of respiration intervals (Pᵢ) is greater than zero and the number of respiration intervals (Nᵢ) is greater than zero, processing circuitry 50 may calculate a respiration interval variation corresponding to Pᵢ and a respiration interval variation corresponding to Nᵢ. If processing circuitry 50 determines that the number of respiration intervals (Nᵢ) is not greater than zero (“NO” branch of block 1206), processing circuitry 50 may determine a respiration interval to be a median of all respiration intervals of the set of respiration intervals (Pᵢ). If, at block 1212, processing circuitry 50 determines that the number of respiration intervals (Nᵢ) is greater than zero (“YES” branch of block 1212), processing circuitry 50 may determine a respiration interval to be a median of all respiration intervals of the set of respiration intervals (Nᵢ) (1214). If processing circuitry 50 determines that the number of respiration intervals (Nᵢ) is not greater than zero (“NO” branch of block 1212), processing circuitry 50 may determine that there is not enough information to determine a respiration interval associated with a respective measurement (1216).

Processing circuitry 50 determines a respiration rate to be equal to 60 seconds/minute divided by the respiration interval in seconds (1218). In this way, the respiration rate may be in units of respiration cycles per minute. In other examples, processing circuitry 50 may calculate the respiration rate to be in other units of measurement such as respiration cycles per second or respiration cycles per hour.

FIG. 11 is a flow diagram illustrating an example operation for determining a peak-to-peak value, in accordance with one or more techniques of this disclosure. For convenience, FIG. 11 is described with respect to IMD 10, external device 12, and processing circuitry 50 of FIGS. 1-2. However, the techniques of FIG. 11 may be performed by different components of IMD 10, external device 12, processing circuitry 50, system 2, or by additional or alternative medical devices.

Processing circuitry 50 may, in some examples, determine a peak-to-peak value indicative of a respiration effort of patient 4 which may represent respiratory parameter information. The peak-to-peak value may represent a signal amplitude, or an approximation of a signal amplitude corresponding to a measurement performed by IMD 10. In order to determine the peak-to-peak value, processing circuitry 50 receives a set of values (1302). Additionally, processing circuitry 50 receives a set of positive zero crossings and a set of negative zero crossings (1304). The set of positive zero crossings and the set of negative zero crossings may, in some examples, be represented by respective values of the set of values. Processing circuitry 50 may determine a group of values following each positive zero crossing (1306) and determine a group of values following each negative zero crossing (1308). The group of values following each respective positive zero crossing may, in some cases, include a positive peak. Additionally, the group of values following each respective negative zero crossing may include a negative peak. In some examples, the group of values following each positive zero crossing is twenty values long. Additionally, in some examples, the group of values following each negative zero crossing is twenty values long.

Processing circuitry 50 identifies a maximum value of the group of values following each positive zero crossing (1310) and identifies a minimum value of the group of value following each negative zero crossing (1312). Processing circuitry 50 calculates a mean maximum value (1314) and calculates a mean minimum value (1316), where the mean maximum value represents a mean value of a set of maximum values corresponding to the set of positive zero crossings and the mean minimum value represents a mean value of a set of minimum values corresponding to the set of negative zero crossings. In other words, the mean maximum value and the mean minimum value may be determined by computing a phase-locked average of positive peaks following positive zero crossings and computing a phase-locked average of negative peaks following negative zero-crossings. For example, phase-locked average may include ensemble averaging, and may be used to determine effort in a respiration signal. Subsequently, processing circuitry 50 calculates a peak-to-peak value (1318) by subtracting the mean minimum value from the mean maximum value. The peak-to-peak value may, in some cases, be indicative of a respiratory effort of patient 4. For example, a greater peak-to-peak value may be associated with a greater respiration effort (i.e., deeper breaths).

Peak-to-peak values are one example of a respiratory parameter indicative of respiratory effort. Another example is an area under the curve (AUC) measurement. To determine AUC, processing circuitry 50 may sum samples of a rectified version of the signal between consecutive positive peaks, negative peaks, positive zero crossings, or negative zero crossings.

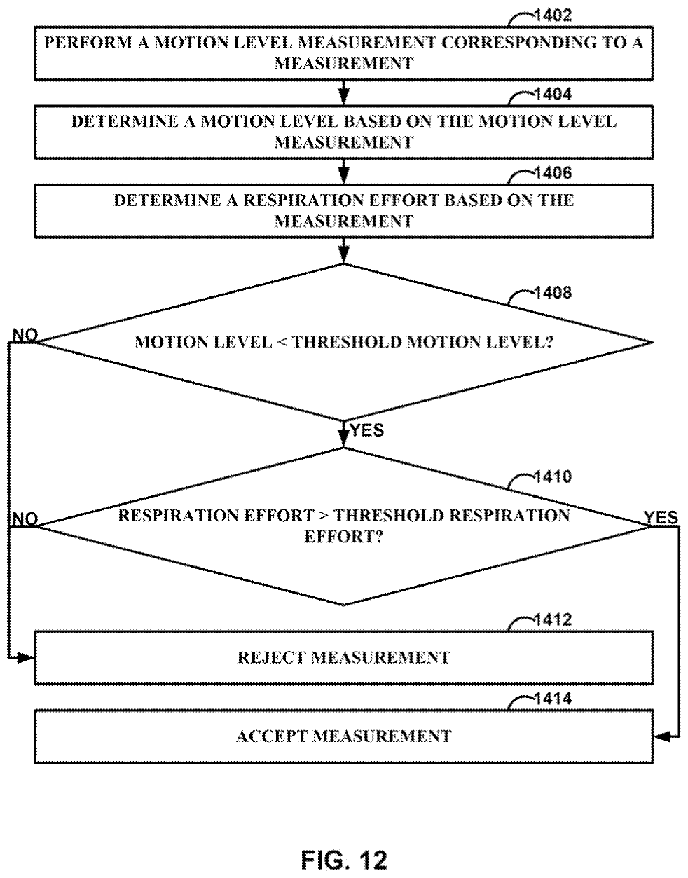

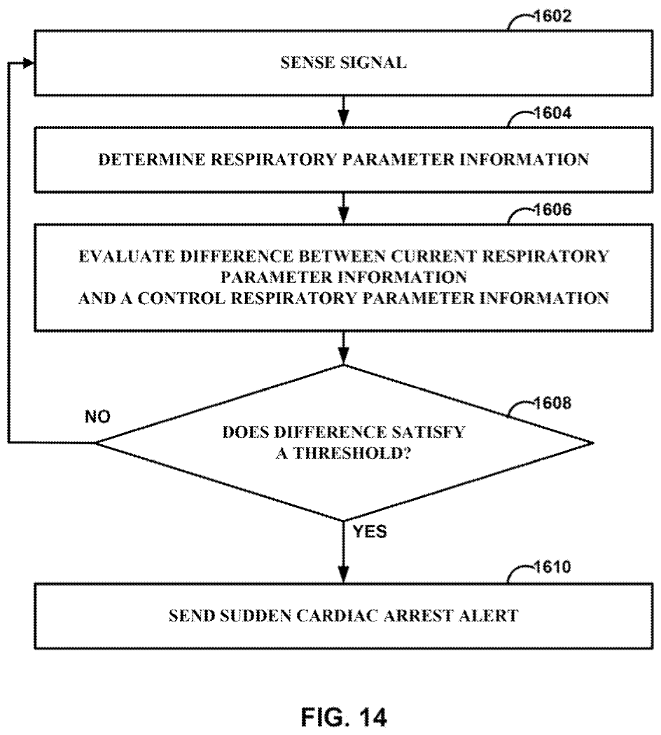

FIG. 12 is a flow diagram illustrating an example operation for determining a quality of a measurement, in accordance with one or more techniques of this disclosure. For convenience, FIG. 12 is described with respect to IMD 10, computing device(s) 12, and processing circuitry 50 of FIGS. 1-2. However, the techniques of FIG. 14 may be performed by different components of IMD 10, external device 12, processing circuitry 50, system 2, or by additional or alternative medical devices.

In some examples, IMD 10, external device 12, processing circuitry 50, or any combination thereof, may evaluate a quality of a measurement. In the example of FIG. 12, ID 10 performs a motion level measurement corresponding to a measurement (1402). For example, IMD 10 may perform the motion level during, shortly before, or shortly after IMD 10 performs the respective measurement in order to obtain data useful for ascertaining a level of motion in patient 4. Processing circuitry 50 determines a motion level based on the motion level measurement (1404). Additionally, processing circuitry 50 determines a respiration effort based on the respective measurement (1406). According to the respiration effort and the motion level, processing circuitry 50 may be configured to evaluate the quality of the measurement.

At block 1408, processing circuitry 50 determines whether a motion level is less than a threshold motion level. If the motion level is not less than the threshold motion level (“NO” branch of block 1408), processing circuitry 50 rejects the measurement (1412). If the motion level is less than the threshold motion level (“YES” branch of block 1408), processing circuitry 50 proceeds to evaluate whether the respiration effort is greater than a threshold respiration effort (1410). If the respiration effort is not greater than a threshold respiration effort (“NO” branch of block 1410), processing circuitry 50 rejects the measurement (1412). If the respiration effort is greater than the threshold respiration effort (“YES” branch of block 1410), processing circuitry 50 accepts the measurement (1414) as a quality measurement. In other words, if the motion level is less than the threshold motion level and the respiration effort is greater than the threshold respiration effort, processing circuitry 50 may determine that conditions are satisfactory such that the measurement may be used for determining respiratory parameters in order to identify or monitor one or more patient conditions (e.g., SCA). Blocks 1408 and 1410 may be performed in any order (e.g., 1408 before 1410 or 1410 before 1408).

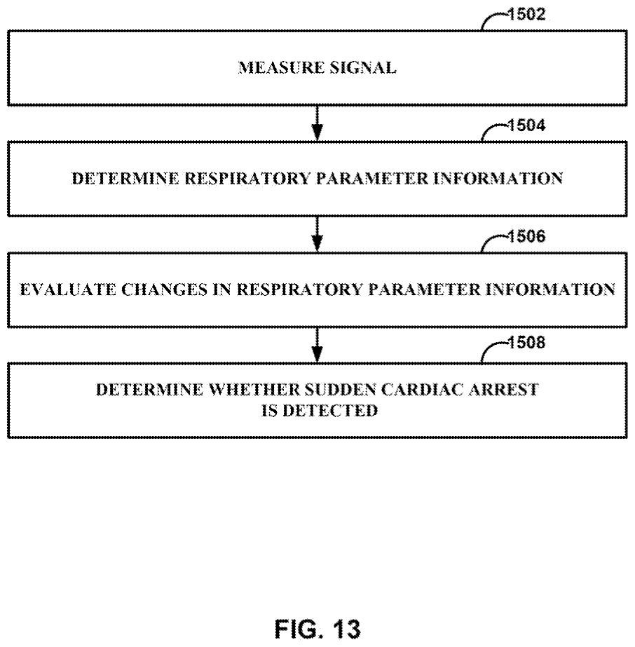

FIG. 13 is a flow diagram illustrating an example operation for determining whether a sudden cardiac arrest is detected, in accordance with one or more techniques of this disclosure. For convenience, FIG. 13 is described with respect to IMD 10, computing device(s) 12, and processing circuitry 50 of FIGS. 1-2. However, the techniques of FIG. 13 may be performed by different components of IMD 10, external device 12, processing circuitry 50, system 2, or by additional or alternative medical devices. Processing circuitry 50 may, in some cases, represent processing circuitry 50 of IMD 10. In such cases, processing circuitry 50 may access memory 52 to obtain stored data or instructions. Additionally, in some cases, processing circuitry 50 represents processing circuitry 80 of external device 12. In such cases, processing circuitry 50 may access memory 52.

One or more sensors or other components may measure a signal of the patient (1502) and obtain a signal value. The signal may be used to determine respiratory parameter information by processing circuitry 50 (1504). In one or more examples, the processor may receive a signal such as, but not limited to an impedance signal, an accelerometer signal, or an electromyogram signal, ECG signal, soundwave signal. The signal may be measured continuously. In some examples, signal measurements are separated by a sampling interval. In other words, the first set of measurements may define a sampling rate. The sampling rate may be within a range between 5 Hz and 16 Hz (e.g., 8 Hz) and a duration of the first set of values may be within a range between 10 seconds and 60 seconds (e.g., 32 seconds). In some examples, the signal may be measured when triggered by an event, such as detection of cardiac arrest based on another physiological parameter, an increase in heart rate of more than 20% over a baseline heart rate, detection of a fast heart rate for a sustained duration (i.e., 32 beats faster than 240 bpm, patient fall, or patient collapse.

In one or more examples, determining respiratory parameter information may include estimating the respiratory effort of the patient, for example, with the processing circuitry 50. In some examples, estimating the respiratory effort comprises determining a peak-to-peak amplitude of the signal for two or more respiratory cycles of the patient. In some examples, estimating the respiratory effort of the patient comprises determining an area under a curve of the signal for at least one respiratory cycle. In one or more examples, determining the respiratory parameter information from the signal comprises averaging, with the processing circuitry, the signal over a plurality of respiratory cycles. In some examples, determining the respiratory parameter information from the signal comprises determining at least one of a respiratory cycle length, inspiratory slope, or expiratory slope. In one or more examples, determining the respiratory parameter information from the signal may include collecting a set of values of the signal, wherein the set of values is indicative of a respiration pattern of a patient, identifying, using the processing circuitry, a set of positive zero crossings based on the set of values, identifying a set of negative zero crossings based on the set of values, determining the respiration effort information using both the set of negative zero crossings and the set of positive zero crossings. In one or more examples, the processing circuitry 50 may determine, for each positive zero crossing of the set of positive zero crossings, a group of values following the respective positive zero crossing, determine, for each negative zero crossing of the set of negative zero crossings, a group of values following the respective negative zero crossing, identify a maximum impedance value of the group of values following each positive zero crossing, identify a minimum impedance value of the group of values following each negative zero crossing, calculate a mean maximum value, calculate a mean minimum value, and calculate a peak-to-peak value by subtracting the mean minimum value from the mean maximum value.

In one or more examples, determining the respiratory parameter information from the signal may include an area under the curve (AUC) measurement. To determine AUC, processing circuitry 50 may sum samples of a rectified version of the signal between consecutive positive peaks, negative peaks, positive zero crossings, or negative zero crossings. In one or more examples, processing circuitry 50 may measure AUC and may compare the current AUC to a previously determined AUC, such as a baseline AUC, or an AUC determined one hour prior to the current AUC, or an AUC determined one day prior to the current AUC. Each difference in AUC may represent a change in respiratory parameter information, and the processing circuitry 50 may evaluate changes in the respiratory parameter information (1506). Using the changes in respiratory parameter information, the processing circuitry 50 may determine whether sudden cardiac arrest is detected (1508). For example, a SCA may be detected based on changes to breathing effort or the manner of breathing of the patient. In some examples, a SCA may be determined by evaluating whether at least one of a difference or a ratio of the current respiratory parameter information and the control respiratory parameter information satisfies a threshold.

FIG. 14 is a flow diagram illustrating another example operation for determining whether a sudden cardiac arrest is detected, in accordance with one or more techniques of this disclosure. For convenience, FIG. 14 is described with respect to IMD 10, computing device 12, and processing circuitry 50 of FIGS. 1-2. However, the techniques of FIG. 14 may be performed by different components of IMD 10, computing device 12, processing circuitry 50, system 2, or by additional or alternative medical devices. The functionality attributed to processing circuitry 50 may, in some examples, be performed in whole or part by processing circuitry 130 of computing device(s) 12.

One or more sensors or other components may measure or sense a signal of the patient (1602) and obtain a signal value. The signal may be used to determine respiratory parameter information by processing circuitry 50 (1604). In one or more examples, the processor may receive a signal such as, but not limited to an impedance signal, an accelerometer signal, or an electromyogram signal. The signal may be measured continuously. The signals may be measured over time and stored to determine a control respiratory parameter information. For example, the control respiratory parameter information may reflect normal breathing effort for a patient. In some examples, the control respiratory parameter information may reflect normal breathing effort for patients of similar age, weight, health backgrounds, etc.

In some examples, signal measurements are separated by a sampling interval. In other words, the first set of measurements may define a sampling rate. The sampling rate may be within a range between 5 Hz and 16 Hz (e.g., 8 Hz) and a duration of the first set of values may be within a range between 10 seconds and 60 seconds (e.g., 32 seconds). In some examples, the signal may be measured when triggered by an event, such as detection of one or more of cardiac arrest, stroke, myocardial infarction, or patient fall, based on another physiological parameter, an increase in heart rate of more than 20% over a baseline heart rate, or patient collapse.

In one or more examples, determining respiratory parameter information may include estimating the respiratory effort of the patient, for example, with the processing circuitry 50. In some examples, estimating the respiratory effort comprises determining a peak-to-peak amplitude of the signal for two or more respiratory cycles of the patient. In some examples, estimating the respiratory effort of the patient comprises determining an area under a curve of the signal for at least one respiratory cycle. In one or more examples, determining the respiratory parameter information from the signal comprises averaging, with the processing circuitry, the signal over a plurality of respiratory cycles. In some examples, determining the respiratory parameter information from the signal comprises determining at least one of a respiratory cycle length, inspiratory slope, or expiratory slope. In one or more examples, determining the respiratory parameter information from the signal may include collecting a set of values of the signal, wherein the set of values is indicative of a respiration pattern of a patient, identifying, using the processing circuitry, a set of positive zero crossings based on the set of values, identifying a set of negative zero crossings based on the set of values, determining the respiration effort information using both the set of negative zero crossings and the set of positive zero crossings. In one or more examples, the processing circuitry 50 may determine, for each positive zero crossing of the set of positive zero crossings, a group of values following the respective positive zero crossing, determine, for each negative zero crossing of the set of negative zero crossings, a group of values following the respective negative zero crossing, identify a maximum value of the group of values following each positive zero crossing, identify a minimum impedance value of the group of values following each negative zero crossing, calculate a mean maximum value, calculate a mean minimum value, and calculate a peak-to-peak value by subtracting the mean minimum value from the mean maximum value.

In one or more examples, determining the respiratory parameter information from the signal may include an area under the curve (AUC) measurement. To determine AUC, processing circuitry 50 may sum samples of a rectified version of the signal between consecutive positive peaks, negative peaks, positive zero crossings, or negative zero crossings. In one or more examples, processing circuitry 50 may measure AUC and may compare the current AUC to a previously determined AUC, such as a baseline AUC, or an AUC determined one hour prior to the current AUC, or an AUC determined one day prior to the current AUC. Each difference in AUC may represent a change in respiratory parameter information, and the processing circuitry 50 may evaluate changes in the respiratory parameter information.

In example examples, the processing circuitry 50 may evaluate a difference between current respiratory parameter information, based upon current sensed signals, and control respiratory parameter information (1606), which may relate to a respiratory parameter information for normal breathing and/or normal breathing effort. In some examples, processing circuitry 50 may categorize respiratory parameter information based on breathing effort for the patient.

A difference between the current respiratory parameter information and the control respiratory parameter information is monitored compared to a threshold value. In some examples, SCA is detected based on a comparison of the current respiratory parameter information and the control respiratory parameter information. In some examples, SCA is detected based on determining whether at least one of a difference or a ratio of the current respiratory parameter information and the control respiratory parameter information satisfies a threshold. If the difference between the current respiratory parameter information and the control respiratory parameter information does not satisfy a threshold (“NO” branch of block 1608), processing circuitry 50 continues to sense signals from the patient (1602). If the difference between the current respiratory parameter information and the control respiratory parameter information does not satisfy a threshold (“YES” branch of block 1608), processing circuitry 50 sends a sudden cardiac arrest alert (1610). In some examples, the alert may be to contact a hospital, summon an ambulance, sound alarm in the patient's residence, alert nearby care providers, contact EMS, e.g., in the United States/North America, use the telephone system to contact a 911 call center, or summon a drone-enabled AED.

Although described herein primarily in the context of time-domain techniques for identifying changes in respiration indicative of SCA, other techniques are contemplated. For example, processing circuitry may receive periodic respiratory parameter information in the form of a digitized respiration signal, e.g., impedance, accelerometer, or EMG signal, and apply the signal or feature vectors derived from the signal to one or more machine learning models. The processing circuitry may determine whether SCA is detected based on an output of the one or more machine learning models.

Example 1. A method comprising: receiving, by a processing circuitry, periodic respiratory parameter information, where the respiratory parameter information includes respiratory effort of a patient; and determining, by the processing circuitry and based on the respiratory parameter information, whether a sudden cardiac arrest of the patient is detected.

Example 2. The method of Example 1, wherein the respiratory parameter information includes respiratory rate of the patient.

Example 3. The method of Example 1 or 2, wherein receiving respiratory parameter information comprises continuously receiving respiratory parameter information.

Example 4. The method of Example 1 or 2, wherein receiving respiratory parameter information comprises receiving respiratory parameter information in response to detection of an event.

Example 5. The method of Example 4, wherein receiving respiratory parameter information in response to detection of the event comprises receiving respiratory parameter information in response to detection of one or more of sudden cardiac arrest, stroke, myocardial infarction, or patient falls based on another physiological parameter.

Example 6. The method of any of Examples 1 to 5, wherein receiving respiratory parameter information comprises: receiving a signal from a sensor; and determining the respiratory parameter information from the signal.

Example 7. The method of Example 6, wherein receiving the signal comprises receiving one or more of an impedance signal, an accelerometer signal, or an electromyogram signal, ECG signal, optical signal, soundwave signal.

Example 8. The method of Example 6 or 7, wherein determining the respiratory parameter information comprises estimating, with the processing circuitry, the respiratory effort of the patient.

Example 9. The method of Example 8, wherein estimating the respiratory effort comprises determining a peak-to-peak amplitude of the signal for two or more respiratory cycles of the patient.

Example 10. The method of Example 8, wherein estimating the respiratory effort of the patient comprises determining an area under a curve of the signal for at least one respiratory cycle.

Example 11. The method of any of Examples 6 to 10, wherein determining the respiratory parameter information from the signal comprises averaging, with the processing circuitry, the signal over a plurality of respiratory cycles.

Example 12. The method of any of Examples 6 to 11, wherein determining the respiratory parameter information from the signal comprises determining at least one of a respiratory cycle length, inspiratory slope, or expiratory slope.

Example 13. The method of any of Example 1 to 12, further comprising comparing a current respiratory parameter information to a control respiratory parameter information, wherein determining whether sudden cardiac arrest is detected comprises determining whether sudden cardiac arrest is detected based on the comparison.

Example 14. The method of Example 13, further comprising determining the control respiratory parameter information from a previous signal.

Example 15. The method of Example 13 or 14, wherein determining whether the sudden cardiac arrest is detected based on the comparison comprises determining whether at least one of a difference or a ratio of the current respiratory parameter information and the control respiratory parameter information satisfies a threshold.

Example 16. The method of any of Examples 6-12, wherein determining the respiratory parameter information from the signal comprises: collecting a set of values of the signal, wherein the set of values is indicative of a respiration pattern of a patient; identifying, using the processing circuitry, a set of positive zero crossings based on the set of values; identifying, using the processing circuitry, a set of negative zero crossings based on the set of values; and determining the respiration effort information using both the set of negative zero crossings and the set of positive zero crossings.

Example 17. The method of Example 16, further comprising: determining, for each positive zero crossing of the set of positive zero crossings, a group of values following the respective positive zero crossing; determining, for each negative zero crossing of the set of negative zero crossings, a group of values following the respective negative zero crossing; identifying a maximum value of the group of values following each positive zero crossing; identifying a minimum value of the group of values following each negative zero crossing; calculating a mean maximum value; calculating a mean minimum value; and calculating a peak-to-peak value by subtracting the mean minimum value from the mean maximum value.

Example 18. The method of Example 1, further comprising categorizing respiratory parameter information based on breathing effort for the patient.

Example 19. The method of any of Examples 1 to 18, further comprising sending an alert based on determining that sudden cardiac arrest is detected.

Example 20. A device comprising: processing circuitry; and memory comprising program instructions that, when executed by the processing circuitry, cause the processing circuitry to: receive periodic respiratory parameter information, where the respiratory parameter information includes respiratory effort of a patient; and determine, based on the respiratory parameter information, whether a sudden cardiac arrest of the patient is detected.

Example 21. The device of Example 20, wherein the respiratory parameter information includes respiratory rate of the patient.

Example 22. The device of Example 20 or 21, wherein the instructions cause the processing circuitry to continuously receive respiratory parameter information.

Example 23. The device of Example 20 or 21, wherein the instructions cause the processing circuitry to receive respiratory parameter information in response to detection of an event.

Example 24. The device of Example 23, wherein the instructions cause the processing circuitry to receive respiratory parameter information in response to detection of sudden cardiac arrest based on another physiological parameter.

Example 25. The device of Examples 20-24, wherein the instructions cause the processing circuitry to: receive a signal from a sensor; and determine the respiratory parameter information from the signal.

Example 26. The device of Example 25, wherein the instructions cause the processing circuitry to receive one or more of an impedance signal, an accelerometer signal, or an electromyogram signal.

Example 27. The device of Example 25 or 26, wherein the instructions cause the processing circuitry to estimate the respiratory effort of the patient based on the signal.

Example 28. The device of Example 27, wherein, to estimate the respiratory effort, the instructions cause the processing circuitry to determine a peak-to-peak amplitude of the signal for two or more respiratory cycles of the patient.

Example 29. The device of Example 27, wherein, to estimate the respiratory effort, the instructions cause the processing circuitry to determine an area under a curve of the signal for at least one respiratory cycle.

Example 30. The device of Examples 25 to 29, wherein to determine the respiratory parameter information from the signal, the instructions cause the processing circuitry to average, with the processing circuitry, the signal over a plurality of respiratory cycles.

Example 31. The device of Examples 25 to 30, wherein to determine the respiratory parameter information from the signal, the instructions cause the processing circuitry to determine at least one of a respiratory cycle length, inspiratory slope, or expiratory slope.

Example 32. The device of any of Examples 20 to 31, wherein the instructions cause the processing circuitry to compare a current respiratory parameter information to a control respiratory parameter information, wherein to determine whether sudden cardiac arrest is detected the instructions cause the processing circuitry to determine whether sudden cardiac arrest is detected based on the comparison.

Example 33. The device of Example 32, wherein the instructions cause the processing circuitry to determine the control respiratory parameter information from a previous signal.

Example 34. The device of Example 32 or 33, wherein to determine whether the sudden cardiac arrest is detected based on the comparison, the instructions cause the processing circuitry to determine whether at least one of a difference or a ratio of the current respiratory parameter information and the control respiratory parameter information satisfies a threshold.

Example 35. The device of any of Examples 25-31, wherein to determine the respiratory parameter information from the signal, the instructions cause the processing circuitry to: collect a set of values of the signal, wherein the set of values is indicative of a respiration pattern of a patient; identify a set of positive zero crossings based on the set of values; identify a set of negative zero crossings based on the set of values; and determine the respiration parameter information using both the set of negative zero crossings and the set of positive zero crossings.

Example 36. The device of Example 35, wherein the instructions cause the processing circuitry to: determine, for each positive zero crossing of the set of positive zero crossings, a group of values following the respective positive zero crossing; determine, for each negative zero crossing of the set of negative zero crossings, a group of values following the respective negative zero crossing; identify a maximum value of the group of values following each positive zero crossing; identify a minimum value of the group of values following each negative zero crossing; calculate a mean maximum value; calculate a mean minimum value; and calculate a peak-to-peak value by subtracting the mean minimum value from the mean maximum value.

Example 37. The device of Example 20, wherein the instructions cause the processing circuitry to categorize respiratory parameter information based on breathing effort for the patient.

Example 38. The device of any of Examples 20-37, wherein the instructions cause the processing circuitry to send an alert based on determining that sudden cardiac arrest is detected.

Example 39. A non-transitory computer-readable medium storing instructions for causing processing circuitry to perform a method comprising: receiving periodic respiratory parameter information, where the respiratory parameter information includes respiratory effort of a patient; and determining, based on the respiratory parameter information, whether a sudden cardiac arrest of the patient is detected.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1-15. (canceled)

16. A method comprising:
receiving, by a processing circuitry, periodic respiratory parameter information in response to detection of an event, where the respiratory parameter information includes respiratory effort of a patient; and
determining, by the processing circuitry and based on the respiratory parameter information, whether a sudden cardiac arrest of the patient is detected.

17. The method of claim 16, wherein the respiratory parameter information includes respiratory rate of the patient.

18. The method of claim 16, wherein receiving respiratory parameter information comprises continuously receiving respiratory parameter information.

19. The method of claim 16, wherein receiving respiratory parameter information in response to detection of the event comprises receiving respiratory parameter information in response to detection of one or more of sudden cardiac arrest, stroke, myocardial infarction, or patient falls based on another physiological parameter.

20. The method of claim 16, wherein receiving respiratory parameter information comprises:
receiving a signal from a sensor; and
determining the respiratory parameter information from the signal.

21. The method of claim 20, wherein receiving the signal comprises receiving one or more of an impedance signal, an accelerometer signal, an electromyogram signal, an electrocardiogram signal, an optical signal, or a soundwave signal.

22. The method of claim 20, wherein determining the respiratory parameter information comprises estimating, with the processing circuitry, the respiratory effort of the patient.

23. The method of claim 22, wherein estimating the respiratory effort comprises determining a peak-to-peak amplitude of the signal for two or more respiratory cycles of the patient.

24. The method of claim 22, wherein estimating the respiratory effort of the patient comprises determining an area under a curve of the signal for at least one respiratory cycle.

25. The method of claim 20, wherein determining the respiratory parameter information from the signal comprises averaging, with the processing circuitry, the signal over a plurality of respiratory cycles.

26. The method of claim 20, wherein determining the respiratory parameter information from the signal comprises determining at least one of a respiratory cycle length, inspiratory slope, or expiratory slope.

27. The method of claim 16, further comprising comparing a current respiratory parameter information to a control respiratory parameter information, wherein determining whether sudden cardiac arrest is detected comprises determining whether sudden cardiac arrest is detected based on the comparison.

28. A device comprising:
processing circuitry; and
memory comprising program instructions that, when executed by the processing circuitry, cause the processing circuitry to:
receive periodic respiratory parameter information in response to detection of an event, where the respiratory parameter information includes respiratory effort of a patient; and
determine, based on the respiratory parameter information, whether a sudden cardiac arrest of the patient is detected.

29. The device of claim 28 wherein the instructions cause the processing circuitry to receive respiratory parameter information in response to detection of one or more of sudden cardiac arrest, stroke, myocardial infarction, or patient falls based on another physiological parameter.

30. The device of claim 28, wherein the instructions cause the processing circuitry to:
receive a signal from a sensor; and
determine the respiratory parameter information from the signal.

31. The device of claim 30, wherein to receive the signal the instructions cause the processing circuitry to receive one or more of an impedance signal, an accelerometer signal, an electromyogram signal, an electrocardiogram signal, an optical signal, or a soundwave signal.

32. The device of claim 31, wherein the instructions cause the processing circuitry to estimate the respiratory effort of the patient based on the one or more of the impedance signal, the accelerometer signal, the electromyogram signal, the electrocardiogram signal, the optical signal, or the soundwave signal.

33. The device of claim 32, wherein, to estimate the respiratory effort, the instructions cause the processing circuitry to determine one or more of a peak-to-peak amplitude of the signal for two or more respiratory cycles of the patient or an area under a curve of the signal for at least one respiratory cycle.

34. The device of claim 30, wherein to determine the respiratory parameter information from the signal, the instructions cause the processing circuitry to perform one or more of:
average the signal over a plurality of respiratory cycles; or
determine at least one of a respiratory cycle length, inspiratory slope, or expiratory slope.

35. A non-transitory computer-readable medium storing instructions for causing processing circuitry to perform a method comprising:
receiving periodic respiratory parameter information in response to detection of an event, where the respiratory parameter information includes respiratory effort of a patient; and
determining, based on the respiratory parameter information, whether a sudden cardiac arrest of the patient is detected.
